(19) United States
(12) Patent Application Publication
(10) Pub. No.: US 2022/0339257 A1
(43) Pub. Date: Oct. 27, 2022

(54) METHODS TO INCREASE THE SENSITIVITY AND REVERSING THE RESISTANCE TO DRUGS

(71) Applicant: Biomarker Strategies, LLC, Rockville, MD (US)

(72) Inventors: Greg P. Bertenshaw, Rockville, MD (US); Meena Chandok, Rockville, MD (US); William Murphy, Rockville, MD (US); Gregory Botting, Rockville, MD (US); Palak Parekh, Rockville, MD (US); Senthamil Selvan, Rockville, MD (US)

(21) Appl. No.: 17/761,461

(22) PCT Filed: Sep. 17, 2020
(86) PCT No.: PCT/US2020/051209

§ 371 Date: Mar. 17, 2022

Related U.S. Application Data
(60) Provisional application No. 62/902,330, filed on Sep. 18, 2019.

(51) Int. Cl. A61K 38/21; A61K 38/19; A61K 38/18; A61K 38/20; A61P 35/00; A61K 31/517; G01N 33/574

(52) U.S. Cl. CPC A61K 38/217; A61K 38/191; A61K 38/1841; A61K 38/204; A61P 35/00; A61K 31/517; G01N 33/574

(57) ABSTRACT

This invention relates to methods of increasing the sensitivity of tumors to anti cancer therapies including antibody therapy, chemotherapy, radiotherapy, and therapies targeting cell signaling pathways such as the MAPK and PI3K pathways and receptor tyrosine kinases (e.g. EGFR). By increasing sensitivity of tumors to these agents, this invention will: (a)Prolong response in already responsive subjects; (b) Increase response rates by converting non-responsive patients into responsive patients; (c) Reverse treatment-induced resistance of tumors to anticancer therapy; and (d) Decrease treatment-associated toxicities by decreasing therapy dosages required for response. The invention also describes predictive tests, to identify patients most likely to respond to the combination treatments.

### CROSS-REFERENCE TO RELATED APPLICATIONS

This application claims benefit of priority under 35 U.S.C. § 119(e) of U.S. Ser. No. 62/902,330, filed Sep. 18, 2019, the entire contents of which is incorporated herein by reference in its entirety.

### GOVERNMENT SUPPORT STATEMENT

This invention was made with government support under Grant Number CA203068 awarded by the National Institutes of Health. The government has certain rights in the invention.

### BACKGROUND OF THE INVENTION

### Field of the Invention

The present invention relates generally to tumor sensitivity and resistance to anticancer therapies, and more specifically to methods of increasing the sensitivity of tumors to anticancer therapies and to predictive testing to identify likelihood of patients' response to therapy.

### Background Information

Molecular characterization of tumors has shifted the paradigm of one drug fits all. Immune checkpoint inhibitors (ICI), molecularly targeted agents (MTAs) and companion/complementary diagnostic tests are being developed to target immune checkpoint proteins, oncogenic drivers in signaling pathways, and select patients most likely to respond, respectively.

The inventors are creating a paradigm shift in combinatorial therapeutic options for cancer patients while advancing the use of ex vivo functional tests in the clinical decision-making process. Our proposed mechanism of overcoming therapy resistance uses novel combinations of therapies and enhancer molecules (EMs). These novel therapeutic regimens will personalize therapy by: (a) Maximizing the survival benefit of first-line treatment in patients with clinically-actionable mutations, (b) Delaying the occurrence of resistance; and (c) Providing options for patients lacking clinically-actionable mutations (i.e. biomarker-negative tumors such as those with wild-type oncogenes). Additionally, we are developing predictive tests, to identify patients most likely to respond to the combination therapies. The test can be deployed using manual methods or optionally enabled using automated instruments such as the SnapPath® Cancer Diagnostics System which automates and standardizes the interrogation of a patient's live tumor cells.

The invention can be utilized in any solid tumor including colorectal, esophageal, stomach, lung, mesothelioma, prostate, uterine, breast, skin, endocrine, melanoma, urinary, pancreas, ovarian, cervical, head and neck, liver, bone, biliary tract, small intestine, hematopoietic, vaginal, testicular, anal, kidney, brain, eye cancer, leukemia, lymphoma, soft tissue, melanoma, and metastases thereof. As way of an example, non-small cell lung cancer (NSCLC) is the most common form of lung cancer and the world's leading cause of cancer death. As such, NSCLC is a highly targeted cancer. The leading MTA treatments for NSCLC inhibit EGFR, with osimertinib (Tagrisso) considered the current standard of care. Other targets include ALK and BRAF/MEK, and there are completed and ongoing clinical trials targeting other proteins in MAPK and PI3K pathways. In the current paradigm of diagnostics, patients are selected for MTA candidacy by the abundance of a protein or the presence of certain gene mutations/rearrangements in their tumors that confer sensitivity to a specific MTA. For example, the presence of activating mutations in EGFR, representing ˜15% of the entire NSCLC population, correlates with the likelihood of response to EGFRi. Intriguingly, a small subset of patients without activating EGFR mutations respond to EGFRi, albeit with weaker responses.

Although various existing treatments can benefit many patients, virtually all patients develop disease recurrence within a year due to clonal expansion of resistant cell clones or emergence of acquired resistance mechanisms making it a significant shortfall in treatment. Thus, even with advances in diagnostics and therapy options, 1- and 5-year survival rates for NSCLC patients with distant metastatic disease, is only 26% and 4%, respectively. Similar poor response times are evident in other cancers. Additionally, many patients lack clinically actionable mutations. For example, most smoker patients, whose tumor onset, and progression are driven by exposure to non-specific mutagens, are considered poor candidates for molecularly targeted agent (MTA) therapy. Therefore, there is a need to better understand primary and acquired resistance mechanisms, improve treatment strategies to address resistance, and develop predictive tests to provide optimal care for patients. Recent progress with MTAs has been the development of next-generation drugs targeting the resistance mechanisms of previous generation therapies. However, resistance mechanisms typically emerge for those and the vicious cycle of fast emerging resistance continues. Resistance is highly complex and similar to the outcome in “multiple drug-resistant bacteria”, making follow-up treatment difficult. There have been many attempts to overcome therapy resistance by using combination therapeutics, but most trials failed to show any clinical advantage. Clearly, to improve survival rates, novel therapeutic strategies are required to prolong response and more accurate predictive tests are needed to address the vast number of patients lacking clinically actionable information.

The best strategy to overcome acquired or innate resistance requires an understanding of tumor-specific pharmacodynamic resistance mechanisms. With this understanding: (a) Molecules can be identified to enhance and prolong responses to improve long-term survival; and (b) New combination therapies can be developed to overcome drug resistance.

Therapies can cause two broad types of responses in tumors: cytotoxic or cytostatic response. In the case of cytotoxic response, the drug actions cause widespread apoptotic and/or necrotic cell death. While in the case of cytostatic responses, the drugs do not remove the tumor in its entirety but rather stagnant growth and progression. Drugs lead to cytostatic effect either by causing cell cycle arrest or senescence. Occasionally, drugs induce a resistance response in tumors as cancer cells try to adapt to the presence of drug and work around it by developing resistance mechanisms. Generally, in the situation of therapeutic attack, cancer cells undergo autophagy to enter a self-protective state. The state of autophagy is a highly reversible process and gives cancer cells an opportunity to revive from an initial therapeutic insult. Our novel combination approach can enhance all types of responses to overcome the autophagy protective mechanism by either further inducing autophagy or reversing autophagy, depending on the biological context.

The second part of current invention relates to concomitantly developing predictive tests to identify patients most likely to respond to the novel combinations. The tests will be superior to existing tests by combining dynamic biomarkers with static biomarkers, as required. The best strategy to achieve this goal requires an understanding of pharmacokinetic and tumor-specific pharmacodynamic resistance mechanisms. Hence, reliable biomarkers that can correctly identify patients which are most likely to experience a favorable or unfavorable response from combination therapies are highly desirable for the success of personalized therapy and overcoming the risks of ineffective treatments which expose patients to undesirable side effects of drugs.

### SUMMARY OF THE INVENTION

The present invention is based on the seminal discovery that modulators (or enhancer molecules, EM) can be used to enhance the response of a subject to an anticancer therapy, and that the likelihood of a subject to respond to a modulator and/or to an anticancer therapy can be predicted.

In one embodiment, the present invention provides methods of enhancing the response of a biological sample/cancer subject to one or more anticancer therapies by using one or more modulators including a) contacting the sample/subject with a modulator prior to, during, simultaneously with, throughout, or following the anticancer therapy to alter the levels, state, or localization of key targets to increase the efficiency of treatment as monotherapy or combination therapy; and b) optionally measuring the levels, state, or localization of key targets and/or related biomarkers in the biological sample/cancer subject to predict response to the therapy and modulator.

In certain aspects, the anticancer therapy and/or modulator are administered by various means selected from the group consisting of intravenous, intraperitoneal, intra/transdermal, intratumoral, subcutaneous, inhalation, ocular, sublingual, epidural, vaginal, intranodal, transmucosal, and rectal routes.

In various aspects, the cancer is colorectal, esophageal, stomach, lung, mesothelioma, prostate, uterine, breast, skin, endocrine, urinary, pancreas, ovarian, cervical, head and neck, liver, bone, biliary tract, small intestine, hematopoietic/blood (myeloma, leukemia, and lymphoma), vaginal, testicular, anal, kidney, brain, eye cancer, leukemia, lymphoma, or soft tissue cancer, melanoma, mixed types, and metastases thereof

In one aspect, the cancer subject is administered a pharmaceutically effective amount of the anticancer therapy and/or modulator. In another aspect, the anticancer agent and/or modulator is formulated with a pharmaceutically acceptable carrier. In many aspects, the anticancer agent and/or modulator is delivered with a delivery system.

In certain aspects, the response and biomarkers are measured in vivo, ex vivo, or in vitro; and the sample is processed for ex vivo or in vitro analysis using one or more manual methods and/or automated systems.

In various aspects, the biological sample is a cancer cell, immune cell, stromal cell, or a subpopulation thereof. In many aspects, the biomarker is measured on tumor cells, stromal cells, immune cells or subpopulation thereof; the tumor cells include cancer stem cells that express one or more of CD133, CD44, ABCG2, and/or ALDH1A1; the stromal cells include fibroblasts; and the immune cells include T cells, B cells, natural killer (NK) cells, dendritic cells, myeloid derived suppressor cells, macrophages, granulocytes, and mast cells. In other aspects, the biological sample is obtained by blood draw, fine needle aspiration, core biopsy, surgical excision, or other tumor sample acquisition method from a model organism or a subject/cancer patient.

In one aspect, the modulator is a phenothiazine, a cytokine, a cannabinoid, a polyphenol, an autophagy modulator, a derivative thereof, a mutant thereof, a peptide thereof, a fragment thereof, an analog thereof or a mimetic thereof. In some aspects, the anticancer therapy is replaced by a modulator.

In certain aspects, the anticancer therapy is a small molecule inhibitor, molecularly targeted agent, antibody, chemical inhibitor, peptide, radiation therapy, chemotherapy, and any other molecule capable of suppressing tumor cell growth.

In various aspects the anticancer agent is an inhibitor of EGFR; ALK one or more members of the VEGF family; KIT; HER2; CDK4 and CDK6; one or more members of the JAK family; mTOR; PI3K/mTOR; AKT; RAS; RAF; MEK; ERK; PDGFRα or PDGFRβ; RET; MET; ROS1; PARP, ATM, PIGF, PTCH, Smoothened, RANKL, and B4GALNT1, (e.g. Ziv-aflibercept, vismodegib, sonidegib, denosumab, dinutuximab); or an antibody targeting PD-1 or PD-1 ligands (PD-L1 or PD-L2).

In other aspects, the anticancer agent is a chemotherapy agent; reactive oxygen species or free radical molecule, a radiation therapy selected from the group consisting of x-rays, gamma rays, or charged particles; or a chromatin modifier.

In one aspect, the biomarker is the localization, and/or level, and/or state of a molecule, and/or organelle. In some aspects, the molecule being measured is a protein or a nucleic acid; the state of the molecule being measured is phosphorylation, acylation, alkylation, amidation, glypiation, glycation, glycosylation, ubiquitination, degradation product(s), truncation, mutation status, or binding of the molecule(s) to promoters; the localization of the molecule being measured is extracellular or cellular, wherein cellular localization includes intracellular, compartmentalized (e.g. Golgi, endoplasmic reticulum, lysosomal, endosomal, exosomal, mitochondrial, vacuole, cytosolic), nuclear or nucleoli, or membrane (e.g. plasma, nuclear and other organelle membranes) bound; and the state of the organelle being observed is nuclear vacuolation/nuclear autophagy, mitophagy.

In another aspect the biomarkers are the direct targets of an anti-cancer agent and/or modulator, wherein the targets include EGFR, ALK, VEGF A, VEGF B, VEGFR, VEGFR1, VEGFR2, VEGFR3, KIT, HER2, CDK4, CDK6, PARP, mTOR, PI3K, AKT1, AKT2, AKT3, n-RAS, k-RAS, c-RAS, a-RAF, b-RAF, c-RAF, MEK1, MEK2, ERK1, ERK2, PDGFRα or PDGFRβ, MET, RET, ROS1, PIGF, PTCH, Smoothened, RANKL, and B4GALNT1, PD-1, PD-L1 and PD-L2.

In other aspects, the biomarkers are STAT1, STAT3, or STAT5; receptor tyrosine kinases; from the PI3K pathway; from the MAPK pathway; or are an immune checkpoint protein.

In another aspect, the biomarkers are markers of apoptosis and necroptosis; markers of cell cycle; markers of proliferation; markers of autophagy, predictive of epithelial mesenchymal transition; are markers of necrosis; or cancer stem cell proteins.

In various aspects, biomarker is Brdu/EdU incorporation and CFSE staining; is the abundance of autophagosomes or autophagolysosomes or is a senescence assay.

In some aspects, a flow cytometry assay is used to measure necrotic, apoptotic and healthy cells.

In one aspect, the biomarker is used to predict resistance is the baseline level of apoptosis markers.

In other aspects, the biomarker(s) are measured using immunoassays, multiplexed assays, PCR, transcription factor assays, nucleic acid or sequencing/mutation testing, cell survival and cell viability assays. In many aspects, the assay is an immunoassay.

In some aspects, the analytical technique is a multiplexed assays.

In various aspects, the analytical technique includes all forms of PCR, including but not limited to qPCR, RT-PCR, real-time PCR and endpoint PCR; is a transcription factor identification assays; includes all forms of sequencing DNA and RNA molecules, whole genome, exome, or specific genes only; and include cell survival and cell viability assay.

In many aspects, the biomarker or panel of biomarkers is used to predict the likelihood of response, wherein the biomarker is selected from the group consisting of EGFR, ALK, VEGF A, VEGF B, VEGFR, VEGFR1, VEGFR2, VEGFR3, KIT, HER2, CDK4, CDK6, PARP, cytochrome c, mTOR, PI3K, AKT1, AKT2, AKT3, n-RAS, k-RAS, c-RAS, a-RAF, b-RAF, c-RAF, MEK1, MEK2, ERK1, ERK2, PDGFRα or PDGFRβ, MET, RET, ROS1, PIGF, PTCH, Smoothened, RANKL, and B4GALNT1, PD-1, PD-L1, PD-L2, STAT1, STAT3, STAT5, HER2, EGFR3, EGFR4, IGF1R, MET, KIT, RET, PDGFR, VEGFR, ALK, BCR-ABL, PTEN, PDK1, S6, p70 S6-Kinase, CREB, GSK3B, mTORC1 and mTORC2, Src, Fak, Ras, Raf, Mek, Erk, CREB, Sos-1, SHC, NFkB, cMyc, ELK-1, c-Fos and c-Jun, caspases 3, 6, 7, 8, 9, PARP, cytochrome c, Bim, Bad, Bax, Bcl-2, Bcl-xL, and Mcl-1, p27 Kip1, cyclin A, cyclin E, cyclin D, cyclin B, CDK1, CDK2, CDK4, CDK6, Cdc2, p16, p21, p14, Ki67, PCNA, LC3I, LC3II, LC3/LC3II/LC3II ratios, MLKL, AMPK, p62/SQSTM1, ATG5-12 complex, ATG13, Vps34, AMBRA-1 and UVRAG, GABA receptor-associated protein like 1, syntaxin-17, LAMP1, LAMP2, LAMP2B, p38, Beclin1, ATM, UNC-51-like kinase-1, -2, and -3, Snail, Slug, Twist, ZEB, vimentin, vinculin, HMGB1, CD133, CD44, ABCG2, ALDH1A1, Brdu/EdU incorporation and CFSE staining, autophagosomes, autophagolysosomes, beta galactosidase, blebbing, Hoechest staining, annexin V staining, propidium iodide staining, WST8/MTT type assays, patient race, gender and age or age-related biomarkers including follicle-stimulating hormone, biopsy type, tumor stage, tumor type and/or histological categorization including cell cycle status, cell type and differentiation status of the sample. In one aspect, the expression levels of biomarkers in a specimen are compared with a non-responsive group and a responsive group of samples. In other aspects, the comparison is performed by a software classification algorithm. In many aspects, the expression levels of the biomarkers are evaluated by applying a statistical method selected from the group consisting of receiver operating characteristic (ROC) curve cut point determination, regression analysis, discriminant analysis, classification tree analysis, random forests, support vector machine, OneR, kNN and heuristic naive Bayes analysis, neural nets and variants thereof.

In an additional aspect, the biological sample/cancer subject is treated in a manner to alter conditions selected from the group consisting of proteins, carbohydrates and lipids, molecules found within fetal bovine serum, growth factors, hypoxia, oxidative stress and physical exercise/stress.

### BRIEF DESCRIPTION OF THE DRAWINGS

FIGS. 1A-1B show the effects of 10 ng/ml EM2 on cell survival measured by MTT assay. FIG. 1A shows the survival of HCC827 cells (NSCLC cells with EGFR deletion mutant) in response to Erlotinib (Erl) and/or EM2 (TNFa). FIG. 1B shows the survival of H1975 cells (NSCLC cells with EGFR double mutant) in response to Erlotinib (Erl) and/or EM2 (TNFa).

FIG. 2 shows the effects of EM2 (TNFa) on H1581 (EGFR WT) cell survival measured by MTT assay. Erl: Erlotinib, 7 μM; 1×: 10 ng/ml; 3×: 30 ng/ml; 5×: 50 ng/ml).

FIG. 3 shows changes in the expression of pERK, pAKT, pGSK, Snail, PDL1, and Bcl-2 biomarkers in an EGFR WT sample (H1581) after sensitizing to erlotinib, evaluated by immunohistochemistry. Mod: enhancer molecule 2 (EM2; TNFa); Erl: Erlotinib; BM: biomarker.

FIG. 4 shows changes in the expression of PDL1, pAKT, Snail, and Bcl-2 biomarkers in HCC827 cells (NSCLC cells with EGFR deletion mutant) and in H1975 cells (NSCLC cells with EGFR double mutant) after sensitizing to erlotinib, evaluated by immunohistochemistry. EM2: enhancer molecule 2 (TNFa); Erl: Erlotinib.

FIG. 5 shows changes in the expression of pERK, pAKT, pGSK, Snail, PDL1, and Bcl-2 biomarkers in a clinical sample in presence and absence of enhancer molecule EM2 (TNFa), evaluated by immunohistochemistry. EM2: enhancer molecule 2 (TNFa); Erl: Erlotinib.

FIG. 6 shows the effect of enhancer molecule EM2 (TNFa) on apoptosis in a clinical sample.

FIGS. 7A-7B show the effect of enhancer molecule EM2 (TNFa) on apoptosis in a clinical sample and in cells using a HoPI Assay. Osi: osimertinib, EM2: enhancer molecule 2 (TNFa). FIG. 7A illustrates the effect of enhancer molecule EM2 (TNFa) on apoptosis in a clinical sample. FIG. 7B illustrates the effect of enhancer molecule EM2 on apoptosis in HS2170 and Calu-6 cells.

FIGS. 8A-8J illustrate examples of enhancement of response to targeted therapies by enhancer molecules. FIG. 8A is a bar graph illustrating enhancement response by IFNg/EM1 in HTB38 cells. FIG. 8B is a bar graph illustrating enhancement response by IFNg/EM1 in OE21 cells. FIG. 8C is a bar graph illustrating enhancement response by IFNg/EM1 in HTB38 cells. FIG. 8D is a bar graph illustrating enhancement response by IFNg/EM1 in HPAF-II cells. FIG. 8E is a bar graph illustrating enhancement response by IFNg/EM1 in CAL27 cells. FIG. 8F is a bar graph illustrating enhancement response by IFNg/EM1 in CALU-6 cells. FIG. 8G is a bar graph illustrating enhancement response by IFNg/EM1 in BICR16 cells. FIG. 8H is a bar graph illustrating enhancement response by IFNg/EM1 in HTHPAF-IIB38 cells. FIG. 8I is a bar graph illustrating enhancement response by IFNg/EM1 in HTB38 cells. FIG. 8J is a bar graph illustrating enhancement response by IFNg/EM1 in BXPC3 cells.

FIGS. 9A-9J illustrate examples of enhancement of response to targeted therapies by enhancer molecules. FIG. 9A is a bar graph illustrating enhancement response by TNFa/EM2 in Calu-6 cells. FIG. 9B is a bar graph illustrating enhancement response by TNFa/EM2 in BICR16 cells. FIG. 9C is a bar graph illustrating enhancement response by TNFa/EM2 in HTB38 cells. FIG. 9D is a bar graph illustrating enhancement response by TNFa/EM2 in A498 cells. FIG. 9E is a bar graph illustrating enhancement response by TNFa/EM2 in RPMI-7951 cells. FIG. 9F is a bar graph illustrating enhancement response by TNFa/EM2 in PC-9 cells. FIG. 9G is a bar graph illustrating enhancement response by TNFa/EM2 in PC-9 Erlotinib-resistant cells. FIG. 9H is a bar graph illustrating enhancement response by TNFa/EM2 in PC-9 Erlotinib-resistant cells. FIG. 9I is a bar graph illustrating enhancement response by TNFa/EM2 in RPMI-7951 cells. FIG. 9J is a bar graph illustrating enhancement response by TNFa/EM2 in OE21 cells.

FIGS. 10A-10D illustrate examples of enhancement of response to targeted therapies by enhancer molecules. FIG. 10A is a bar graph illustrating enhancement response by TGFb/EM3 in SK-MEL-28 cells. FIG. 10B is a bar graph illustrating enhancement response by TGFb/EM3 in Calu-6 cells. FIG. 10C is a bar graph illustrating enhancement response by TGFb/EM3 in SK-MEL-28 cells. FIG. 10D is a bar graph illustrating enhancement response by TGFb/EM3 in SK-MES-1 cells.

FIGS. 11A-11E illustrate examples of enhancement of response to targeted therapies by enhancer molecules. FIG. 11A is a bar graph illustrating enhancement response by IL17/EM5 in HPAF-II cells. FIG. 11B is a bar graph illustrating enhancement response by IL17/EM5 in Calu-6 cells. FIG. 11C is a bar graph illustrating enhancement response by IL17/EM5 in A253 cells. FIG. 11D is a bar graph illustrating enhancement response by IL17/EM5 in Ca9-22 cells. FIG. 11E is a bar graph illustrating enhancement response by IL17/EM5 in H2170 cells.

FIGS. 12A-12C illustrate examples of enhancement of response to targeted therapies by enhancer molecules. FIG. 12A is a bar graph illustrating enhancement response by TNFb/EM6 in Ca9-22 cells. FIG. 12B is a bar graph illustrating enhancement response by TNFb/EM6 in Calu-6 cells. FIG. 12C is a bar graph illustrating enhancement response by TNFb/EM6 in H2170 cells.

FIGS. 13A-13V illustrate examples of enhancement of response to targeted therapies by enhancer molecules. FIG. 13A is a bar graph illustrating enhancement response by IFNa/EM7 in A7 cells. FIG. 13B is a bar graph illustrating enhancement response by IFNa/EM7 in Ca9-22 cells. FIG. 13C is a bar graph illustrating enhancement response by IFNa/EM7 in PE/CA-PJ15 cells. FIG. 13D is a bar graph illustrating enhancement response by IFNa/EM7 in A7 cells. FIG. 13E is a bar graph illustrating enhancement response by IFNa/EM7 in A7 cells. FIG. 13F is a bar graph illustrating enhancement response by IFNa/EM7 in Ca9-22 cells. FIG. 13G is a bar graph illustrating enhancement response by IFNa/EM7 in A7 cells. FIG. 13H is a bar graph illustrating enhancement response by IFNa/EM7 in Ca9-22 cells. FIG. 13I is a bar graph illustrating enhancement response by IFNa/EM7 in H1838 cells. FIG. 13J is a bar graph illustrating enhancement response by IFNa/EM7 in H1838 cells. FIG. 13K is a bar graph illustrating enhancement response by IFNa/EM7 in PE/CA-PJ15 cells. FIG. 13L is a bar graph illustrating enhancement response by IFNa/EM7 in Ca9-22 cells. FIG. 13M is a bar graph illustrating enhancement response by IFNa/EM7 in H2170 cells. FIG. 13N is a bar graph illustrating enhancement response by IFNa/EM7 in PE/CA-PJ15 cells. FIG. 13O is a bar graph illustrating enhancement response by IFNa/EM7 in A7 cells. FIG. 13P is a bar graph illustrating enhancement response by IFNa/EM7 in Ca9-22 cells. FIG. 13Q is a bar graph illustrating enhancement response by IFNa/EM7 in A7 cells. FIG. 13R is a bar graph illustrating enhancement response by IFNa/EM7 in A7 cells. FIG. 13S is a bar graph illustrating enhancement response by IFNa/EM7 in Calu-6 cells. FIG. 13T is a bar graph illustrating enhancement response by IFNa/EM7 in A7 cells. FIG. 13U is a bar graph illustrating enhancement response by IFNa/EM7 in A427 cells. FIG. 13V is a bar graph illustrating enhancement response by IFNa/EM7 in Ca9-22 cells.

FIG. 14 is a bar graph illustrating enhancement response by IL-12/EM9 in PE/CA-PJ15 cells.

FIGS. 15A-15D illustrate examples of enhancement of response to targeted therapies by enhancer molecules. FIG. 15A is a bar graph illustrating enhancement response by GM-CSF/EM10 in H358 cells. FIG. 15B is a bar graph illustrating enhancement response by GM-CSF/EM10 in A498 cells. FIG. 15C is a bar graph illustrating enhancement response by GM-CSF/EM10 in A253 cells. FIG. 15D is a bar graph illustrating enhancement response by GM-CSF/EM10 in PE/CA-PJ15 cells.

FIGS. 16A-16B illustrates unique Ki67 degradation patterns predictive of response to therapies analyzed by western blot. FIG. 16A shows immunoblots illustrating the changes of expression levels and degradation patterns of Ki67 and PARP in strong-responder and in non-responder. FIG. 16B shows bar graphs quantifying cell survival.

FIGS. 17A-17D illustrate predictive biomarkers for EM+MTA response analyzed by western blot. FIG. 17A shows immunoblots illustrating the expression levels of Ki67, PARP, LC3, p62, and pAKT in HTB-38, RPMI-7951, Calu-6 and HPAF-II cells. FIG. 17B shows immunoblots illustrating the expression levels of Ki67, PARP, LC3, p62, pERK, and pAKT in PE/CA-PJ15, CAL27, BICR-16 and PC-9 cells. FIG. 17C shows immunoblots illustrating the expression levels of Ki67, PARP, LC3, p62, and pAKT in H1838, A7, A-253 and PE/CA-PJ15cells. FIG. 17D shows immunoblots illustrating the expression levels of Ki67, PARP, LC3, p62, and pAKT Ca9-22 cells.

FIG. 18 illustrates HoPI data analyzed in RPMI-7951, Ca9-22, HTB38 and PE/CA-JP15 cells.

### DETAILED DESCRIPTION OF THE INVENTION

The present invention is based on the seminal discovery that modulators (or enhancer molecules) can be used to enhance the response of a subject to an anticancer therapy.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

In one embodiment, the present invention provides method of enhancing the response of a biological sample/cancer subject to one or more anticancer therapies by using one or more modulators including a) contacting the sample/subject with a modulator prior to, during, simultaneously with, throughout, or following the anticancer therapy to alter the levels, state, or localization of key targets to increase the efficiency of treatment as monotherapy or combination therapy; and b) optionally measuring the levels, state, or localization of key targets and/or related biomarkers in the biological sample/cancer subject to predict response to the therapy and modulator.

The term “enhancing the response” refers to both the amelioration of the results observed with the combination of an anti-cancer therapy with a modulator compared to the anti-cancer therapy alone; and to the ability of a modulator to re-sensitize a non-responsive tumor to an anti-cancer therapy.

The term “subject” as used herein refers to any individual or patient to which the subject methods are performed. Generally, the subject is human, although as will be appreciated by those in the art, the subject may be an animal. Thus other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject. “Cancer subject” refers to the individual, to which the subject methods are performed, which has a tumor or cancer.

The term “cancer” refers to a group of diseases characterized by abnormal and uncontrolled cell proliferation starting at one site (primary site) with the potential to invade and to spread to others sites (secondary sites, metastases) which differentiate cancer (malignant tumor) from benign tumor. Virtually all the organs can be affected, leading to more than 100 types of cancer that can affect humans. Cancers can result from many causes including genetic predisposition, viral infection, exposure to ionizing radiation, exposure environmental pollutant, tobacco and or alcohol use, obesity, poor diet, lack of physical activity or any combination thereof. As used herein, “neoplasm” or “tumor” including grammatical variations thereof, means new and abnormal growth of tissue, which may be benign or cancerous. In a related aspect, the neoplasm is indicative of a neoplastic disease or disorder, including but not limited, to various cancers. For example, such cancers can include prostate, pancreatic, biliary, colon, rectal, liver, kidney, lung, testicular, breast, ovarian, pancreatic, brain, and head and neck cancers, melanoma, sarcoma, multiple myeloma, leukemia, lymphoma, and the like.

“Anti-cancer therapy”, as used herein, is meant to refer to any chemotherapeutic agent, any anti-neoplasic agent, to radiation, or to any substance or agent known in the art to have a toxic effect on cells resulting in the death of cancer cells regardless of the cellular pathway leading to it. The anticancer therapies used in the present invention might be used alone or in combination with one another.

The term “treatment” is used interchangeably herein with the term “therapeutic method” and refers to both 1) therapeutic treatments or measures that cure, slow down, lessen symptoms of, and/or halt progression of a diagnosed pathologic conditions or disorder, and 2) and prophylactic/preventative measures. Those in need of treatment may include individuals already having a medical disorder as well as those who may ultimately acquire the disorder (i.e., those needing preventive measures).

The phrases “combination therapy”, “combined with” and the like refer to the use of more than one medication or treatment simultaneously to increase the response.

As used herein “enhancer molecule”, “EMs”, or “modulator” are used interchangeably, and refer to any molecules that can be used to enhance the response of a subject to an anti-cancer therapy, and/or to induce a response in a subject that is resistant to an anti-cancer therapy.

As used herein “biological sample” refers to any cancer sample collected from a subject having a tumor, regardless or the site of origin. Biological samples can include, but are not limited to biopsy, surgical resection, blood, plasma, saliva, or any sample collected from a patient that is susceptible to contain cancer cells.

In certain aspects, the anticancer therapy and/or modulator are administered by various means including intravenous, intraperitoneal, intra/transdermal, intratumoral, subcutaneous, inhalation, ocular, sublingual, epidural, vaginal, intranodal, transmucosal, and rectal routes.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticular, subcapsular, subarachnoid, intraspinal and intrasternal, oral, sublingual buccal, rectal, vaginal, nasal ocular administrations, as well infusion, inhalation, and nebulization. The phrases “parenteral administration” and “administered parenterally” as used herein means modes of administration other than enteral and topical administration.

In various aspects, the cancer is colorectal, esophageal, stomach, lung, mesothelioma, prostate, uterine, breast, skin, endocrine, urinary, pancreas, ovarian, cervical, head and neck, liver, bone, biliary tract, small intestine, hematopoietic/blood (myeloma, leukemia, and lymphoma), vaginal, testicular, anal, kidney, brain, eye cancer, leukemia, lymphoma, or soft tissue cancer, melanoma, mixed types, and metastases thereof.

In one aspect, the cancer subject is administered a pharmaceutically effective amount of the anticancer therapy and/or modulator.

The terms “therapeutically effective amount”, “effective dose,” “therapeutically effective dose”, “effective amount,” or the like refer to that amount of the subject compound that will elicit the biological or medical response of a tissue, system, animal or human that is being sought by the researcher, veterinarian, medical doctor or other clinician.

In another aspect, the anticancer agent and/or modulator is formulated with a pharmaceutically acceptable carrier.

By “pharmaceutically acceptable” it is meant that the carrier, diluent or excipient must be compatible with the other ingredients of the formulation and not deleterious to the recipient thereof. Pharmaceutically acceptable carriers, excipients or stabilizers are well known in the art, for example Remington's Pharmaceutical Sciences, 16th edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG).

In many aspects, the anticancer agent and/or modulator is delivered with a delivery system.

“Delivery systems” are well known in the art and refer to any means that can be used to deliver an agent of interest to a target site. For example, delivery system may include syringe, needle, and catheter.

The mode of delivery for the administration of EMs and/or anticancer therapies according to the present invention to a subject, such as a human patient or mammalian animal, will depend in large part on the particular active agent present and the target cells. Methods for the targeted delivery of drugs or agents are well known in the art, and include but are not limited to the use of targeted antibodies; targeting moiety (i.e., a molecule that has the ability to localize and bind to a specific molecule or cellular component, such as an antibody, antibody fragment, scFv, Fc-containing polypeptide, fusion antibody, polypeptide, peptide, aptamer, ligand, nucleic acid, or any combination thereof); nanospheres; and nanoparticles, including coated nanoparticles like PEGylated nanoparticles or antibody coated nanoparticles. As used herein, “targeted delivery” is meant to refer to any means that help deliver the modulator and/or therapy to the tumor, or target site or organ.

In certain aspects, the response and biomarkers are measured in vivo, ex vivo, or in vitro; and the sample is processed for ex vivo or in vitro analysis using one or more manual methods and/or automated systems.

In various aspects, the biological sample is a cancer cell, immune cell, stromal cell, or subpopulation thereof. In many aspects, the biomarker is measured on tumor cells, stromal cells, immune cells or subpopulation thereof; the tumor cells include cancer stem cells that express one or more of CD133, CD44, ABCG2, and/or ALDH1A1; the stromal cells include fibroblasts; and the immune cells include T cells, B cells, natural killer (NK) cells, dendritic cells, myeloid derived suppressor cells, macrophages, granulocytes, and mast cells.

As used herein, the term “cancer stem cell” refers to cells found within tumors or hematological cancers that possess characteristics associated with normal stem cells, specifically the ability to give rise to all cell types found in a particular cancer sample, and the self-renewal properties. Therefore, such cells are hypothesized to persist in tumors as a distinct population and cause relapse and metastasis by giving rise to new tumors; and the development of specific therapies targeting cancer stem cells holds hope for improvement of survival and quality of life of cancer patients, especially for patients with metastatic disease. Several biomarkers have been identified as specific cancer stem cell biomarkers. Examples of biomarkers of cancer stem cells include CD133, CD44, ABCG2, and/or ALDH1A1.

In some aspects, the biological sample is obtained by blood draw, fine needle aspiration, core biopsy, surgical excision, or other tumor sample acquisition method from a model organism or a subject/cancer patient.

In one aspect, the modulator is a phenothiazine, a cytokine, a cannabinoid, a polyphenol, an autophagy modulator, a derivative thereof, a mutant thereof, a peptide thereof, a fragment thereof, an analog thereof or a mimetic thereof.

As used herein, “phenothiazine” refers to an antipsychotic drug. Antipsychotics can be used to reduce hallucinations and delusions associated with psychosis. Some phenothiazines (such as prochlorperazine and chlorpromazine) are also effective at relieving other symptoms unrelated to psychosis, such as nausea, vomiting, prolonged hiccups, tetanus symptoms and hyper-excitable behavior in children. Non-limiting examples of phenothiazine include: prochlorperazine, chlorpromazine, trifluoperazine, fluphenazine, thioridazine, perphenazine and mesoridazine.

As used herein, “cytokine” refers to small proteins (˜5-20 kDa) that are important in cell signaling, as well as to any derivative thereof, mutant thereof, peptide thereof, fragment thereof, analog thereof or mimetic thereof. Cytokines are involved in autocrine signaling, paracrine signaling and endocrine signaling as immunomodulating agents. They include chemokines, interferons, interleukins, lymphokines, and tumor necrosis factors.

As used herein “cannabinoid” refers diverse chemical compounds that act on cannabinoid receptors. Ligands for these receptor proteins include the endocannabinoids produced naturally in the body by animals; phytocannabinoids, found in cannabis; and synthetic cannabinoids, manufactured artificially. The most notable cannabinoid is the phytocannabinoid tetrahydrocannabinol (THC), the primary psychoactive compound in cannabis. Cannabidiol (CBD) is another major constituent of the plant. There are at least 113 different cannabinoids isolated from cannabis.

As used herein “polyphenol” refers to mainly natural, but also synthetic or semisynthetic, organic chemicals characterized by the presence of large multiples of phenol structural units.

The term “autophagy” refers to the natural, regulated mechanism of the cell that removes unnecessary or dysfunctional components. It allows the orderly degradation and recycling of cellular components. As used herein, “autophagy modulator” refers to any compound that can modulate (inhibit or enhance) autophagy.

In other aspects, the phenothiazine is prochlorperazine, the cytokine is interferon (IFN) gamma, tumor necrosis factor (TNF) alpha, tumor growth factor (TGF) beta, interleukin (IL) 6, or IL-17; the cannabinoid is synthetic THC-Delta-9 THC or cannabidiol (CBD); the polyphenol is resveratrol or RSVAs; and the autophagy modulator is metformin, melatonin, trehalose, spermidine, spermine, azithromycin, chloroquine, or chloramphenicol. In some aspects, the anticancer therapy is replaced by a modulator.

In certain aspects, the anticancer therapy is small molecule inhibitor, a molecularly targeted agent, antibody, chemical inhibitor, peptide, reactive oxygen species or free radical molecule, radiation therapy, chemotherapy and any other molecule capable of suppressing tumor cell growth.

As used herein the terms “Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. “Antibody,” as used herein, encompasses any polypeptide including an antigen-binding site regardless of the source, species of origin, method of production, and characteristics. Antibodies include natural or artificial, mono- or polyvalent antibodies including, but not limited to, polyclonal, monoclonal, multispecific, human, humanized or chimeric antibodies, single chain antibodies, and antibody fragments. “Antibody fragments” include a portion of an intact antibody, preferably the antigen binding or variable region of the intact antibody. Examples of antibody fragments include Fab, Fab′ and F(ab′)2, Fc fragments or Fc-fusion products, single-chain Fvs (scFv), disulfide-linked Fvs (sdfv) and fragments including either a VL or VH domain; diabodies, tribodies and the like (Zapata et al. Protein Eng. 8(10):1057-1062 [1995]).

The term “antibody,” as used herein, refers to immunoglobulin molecules and immunologically active portions of immunoglobulin molecules, i.e., molecules that contain an antigen binding site that immunospecifically binds an antigen. “Native antibodies” and “intact immunoglobulins”, or the like, are usually heterotetrameric glycoproteins of about 150,000 daltons, composed of two identical light (L) chains and two identical heavy (H) chains. The light chains from any vertebrate species can be assigned to one of two clearly distinct types, called kappa (κ) and lambda (λ), based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of immunoglobulins are called α, δ, ε, γ, and μ, respectively. The subunit structures and three-dimensional configurations of different classes of immunoglobulins are well known.

The intact antibody may have one or more “effector functions” which refer to those biological activities attributable to the Fc region (a native sequence Fc region or amino acid sequence variant Fc region or any other modified Fc region) of an antibody. Examples of antibody effector functions include Clq binding; complement dependent cytotoxicity; Fc receptor binding; antibody-dependent cell-mediated cytotoxicity (ADCC); phagocytosis; down regulation of cell surface receptors (e.g., B cell receptor (BCR); and cross-presentation of antigens by antigen presenting cells or dendritic cells.

As used herein, “reactive oxygen species” or “ROS” refers to chemically reactive chemical species containing oxygen. ROS are formed as a natural byproduct of the normal metabolism of oxygen and have important roles in cell signaling and homeostasis. However, during times of environmental stress or oxidative stress (e.g., UV or heat exposure), ROS levels can increase dramatically. This may result in significant damage to cell structures. Examples of ROS include peroxides, superoxide, hydroxyl radical, singlet oxygen, and alpha-oxygen.

“Chemotherapy”, “chemotherapeutic and antineoplastic agents” are used interchangeably and refer to well-known cytotoxic agents, that can be used to treat cancer; they include: (i) anti-microtubules agents including vinca alkaloids (vinblastine, vincristine, vinflunine, vindesine, and vinorelbine), taxanes (cabazitaxel, docetaxel, larotaxel, ortataxel, paclitaxel, and tesetaxel), epothilones (ixabepilone), and podophyllotoxin (etoposide and teniposide); (ii) antimetabolite agents including anti-folates (aminopterin, methotrexate, pemetrexed, pralatrexate, and raltitrexed), and deoxynucleoside analogues (azacitidine, capecitabine, carmofur, cladribine, clofarabine, cytarabine, decitabine, doxifluridine, floxuridine, fludarabine, fluorouracil, gemcitabine, hydroxycarbamide, mercaptopurine, nelarabine, pentostatin, tegafur, and thioguanine); (iii) topoisomerase inhibitors including Topoisomerase I inhibitors (belotecan, camptothecin, cositecan, gimatecan, exatecan, irinotecan, lurtotecan, silatecan, topotecan, and rubitecan) and Topoisomerase II inhibitors (aclarubicin, amrubicin, daunorubicin, doxorubicin, epirubicin, etoposide, idarubicinm, merbarone, mitoxantrone, novobiocin, pirarubicin, teniposide, valrubicin, and zorubicin); (iv) alkylating agents including nitrogen mustards (bendamustine, busulfan, chlorambucil, cyclophosphamide, estramustine phosphate, ifosamide, mechlorethamine, melphalan, prednimustine, trofosfamide, and uramustine), nitrosoureas (carmustine (BCNU), fotemustine, lomustine (CCNU), N-Nitroso-N-methylurea (MNU), nimustine, ranimustine semustine (MeCCNU), and streptozotocin), platinum-based (cisplatin, carboplatin, dicycloplatin, nedaplatin, oxaliplatin and satraplatin), aziridines (carboquone, thiotepa, mytomycin, diaziquone (AZQ), triaziquone and triethylenemelamine), alkyl sulfonates (busulfan, mannosulfan, and treosulfan), non-classical alkylating agents (hydrazines, procarbazine, triazenes, hexamethylmelamine, altretamine, mitobronitol, and pipobroman), tetrazines (dacarbazine, mitozolomide and temozolomide); (v) anthracyclines agents including doxorubicin and daunorubicin. Derivatives of these compounds include epirubicin and idarubicin; pirarubicin, aclarubicin, and mitoxantrone, bleomycins, mitomycin C, mitoxantrone, and actinomycin; (vi) enzyme inhibitors agents including FI inhibitor (Tipifarnib), CDK inhibitors (Abemaciclib, Alvocidib, Palbociclib, Ribociclib, and Seliciclib), PrI inhibitor (Bortezomib, Carfilzomib, and Ixazomib), PhI inhibitor (Anagrelide), IMPDI inhibitor (Tiazofurin), LI inhibitor (Masoprocol), PARP inhibitor (Niraparib, Olaparib, Rucaparib), HDAC inhibitor (Belinostat, Panobinostat, Romidepsin, Vorinostat), and PIKI inhibitor (Idelalisib); (vii) receptor antagonist agent including ERA receptor antagonist (Atrasentan), Retinoid X receptor antagonist (Bexarotene), Sex steroid receptor antagonist (Testolactone); (viii) ungrouped agent including Amsacrine, Trabectedin, Retinoids (Alitretinoin Tretinoin) Arsenic trioxide, Asparagine depleters (Asparaginase/Pegaspargase), Celecoxib, Demecolcine Elesclomol, Elsamitrucin, Etoglucid, Lonidamine, Lucanthone, Mitoguazone, Mitotane, Oblimersen, Omacetaxine mepesuccinate, and Eribulin.

In various aspects the anticancer agent is an inhibitor of epidermal growth factor receptor (EGFR, e.g. erlotinib, cetuximab, osimertinib, vandetanib, panitumumab, necitumumab, gefitinib and afatinib); Anaplastic lymphoma kinase (ALK, e.g. alectinib, brigatinib, ceritinib, and crizotinib); one or more members of the Vascular endothelial growth factor (VEGF) family (VEGF ligand, VEGFR, VEGFR2, VEGFA/B, VEGFR1/2/3; e.g. bevacizumab, pazopanib, ramucirumab, sorafenib, Ziv-aflibercept, lenvatinib, axitinib, vandetanib, cabozantinib, and regorafenib); KIT (e.g. axitinib, cabozantinib, imatinib, pazopanib, regorafenib); human epidermal growth factor receptor 2 (HER2, e.g. lapatinib, neratinib, pertuzumab, dacomitinib, trastuzumab and ado-trastuzumab emtansine); Cyclin-dependent kinase (CDK4 and CDK6, e.g. palbociclib and ribociclib); Poly (ADP-ribose) polymerase (PARP, e.g. niraparib, olaparib, and rucaparib); one or more members of the Janus kinases (JAK family, e.g. ruxolitinib and tofacitinib); mammalian target of rapamycin (mTOR) via direct inhibition or indirectly via binding of FK-binding protein 12 (e.g. omipalisib, dactolisib, pictilisib, idelalisib, buparlisib, torins, rapamycin, everolimus and temsirolimus); Phosphoinositide 3-kinases (PI3K)/mTOR (e.g. omipalisib, dactolisib, pictilisib, idelalisib and buparlisib, 3 methyl adenine, wortmannin); Protein kinase B (PKB or AKT, e.g. uprosertib, MK-2206, ipatasertib, capivasertib and ARQ092); RAS (e.g. sulindac, IND12, ARS-1620, BIM-46187, ARS853, 3144 (Pan-Ras inhibitor), 4AM, 9A5); RAF proteins (e.g. regorafenib, sorafenib, dabrafenib and vemurafenib); Mitogen-activated protein kinase kinase (MEK, e.g. cobimetinib and trametinib); extracellular signal-regulated kinases (ERK, e.g. ulixertinib); platelet-derived growth factor receptor (PDGFRα or PDGFRβ, e.g. axitinib, imatinib, olaratumab, pazopanib, regorafenib, and sorafenib); RET (e.g. regorafenib, vandetanib and cabozantinib); MET (e.g. cabozantinib and crizotinib); ROS1 (e.g. crizotinib); PARP, ATM, PIGF, PTCH, Smoothened, RANKL, and B4GALNT1, (e.g. Ziv-aflibercept, vismodegib, sonidegib, denosumab, dinutuximab); or an antibody targeting PD-1 or PD-1 ligands (PD-L1 or PD-L2); STAT family (e.g. danvatirsen, AZD9150 and TTI-101); MAC (including but not limited to HDAC3 and HDAC6) family, (e.g. nexturastat A, ricolinostat, trichostatin A, vorinostat, panobinostat, vaproic acid, belinostat, and entinostat); BET family (including but not limited to BRD1 and BRD4) (e.g RG6146, ABBV-075, OTX015/MK-8628, GSK2820151/I-BET151, CC-90010, PLX51107, and LY294002); NTRK (e.g. larotrectinib and entrectinib); Bcl-2 (e.g. venetoclax, navitoclax, and obatoclax); ATM (e.g. AZD1390, AZD0156, and M3541); ATR (e.g. AZD6738 and M6620); A2AR (e.g. AZD4635 and CPI-444); WEE1 (e.g. adavosertib and ADZ1775); FGFR (e.g. erdafitinib, pemigatinib and TAS-120).

In other aspects, the anticancer agent is a chemotherapy agent (e.g. paclitaxel, gemcitabine, doxorubicin, cisplatin, and others); reactive oxygen species or a free radical molecule, a radiation therapy such as x-rays, gamma rays, or charged particles; or a chromatin modifier (e.g. romidepsin, 5-azacytidine, decitabine, suberolanilide hydroxamic acid (SAHA), belinostat, panobinostat).

As used herein “chromatin modifiers” refer to any agent that is capable of inducing chromatin modification or remodeling.

In some aspects, the present invention provides for combination therapy, that may include one of more EMs, combines with one or more anticancer agents.

Generic examples of tri-therapies, including one EM and two anticancer therapies, or two EMs and one anticancer therapy include, but are not limited to: EM1/RTK/MAPK, EM1/RTK/PI3K, EM1/MAPK/PI3K, EM1/EM2/RTK, EM1/EM3/RTK, EM1/EM4/RTK, EM2/EM3/RTK, EM2/EM4/RTK, EM3/EM4/RTK, EM1/EM2/PI3K, EM1/EM3/PI3K, EM1/EM4/PI3K, EM2/EM3/PI3K, EM2/EM4/PI3K, EM3/EM4/PI3K, EM1/EM2/MAPK, EM1/EM3/MAPK, EM1/EM4/MAPK, EM2/EM3/MAPK, EM2/EM4/MAPK, EM3/EM4/MAPK where RTK is any receptor tyrosine kinase inhibitor, MAPK is any inhibitor of the MAPK pathway, and where PI3K is any inhibitor of the PI3K pathway.

Specific examples of tri-therapies, including one EM and two anticancer therapies, or two EMs and one anticancer therapy include, but are not limited to: EM1/Alectinib/Linsitinib, EM1/Alectinib/GSK-458, EM1/GSK-458/Linsitinib, EM1/Dabrafenib/Everolimus, EM1/Dabrafenib/GSK-795, EM1/GSK-795/Everolimus, EM1/Linsitinib/Dabrafenib, EM1/Linsitinib/Everolimus, EM1/Linsitinib/GSK-795, EM1/Linsitinib/GSK-458, EM1/GSK-795/GSK-458, EM1/Everolimus/GSK-458, EM1/Osimertinib/Dabrafenib, EM1/Osimertinib/Trametinib, EM2/Cetuximab/Dabrafenib, EM2/Cetuximab/Osimertinib, EM2/Cetuximab/Trametinib, EM2/Cetuximab/GSK-795, EM2/Cetuximab/GSK-458, EM2/Cetuximab/Erlotinib, EM2/Cetuximab/alectinib, EM2/Cetuximab/everolimus, EM2/Cetuximab/Lisitinib, EM2/Dabrafenib/Everolimus, EM2/Dabrafenib/GSK-458, EM2/Dabrafenib/GSK-795, EM2/Dabrafenib/Trametinib, EM2/EM1/Cetuximab, EM2/EM1/Dabrafenib, EM2/EM1/Everolimus, EM2/EM1/GSK-795, EM2/EM1/Linsitinib, EM2/EM1/Osimertinib, EM2/EM1/Erlotinib, EM2/EM1/Alectinib, EM2/EM1/GSK-458, EM2/EM1/Trametinib, EM2/GSK-795/Everolimus, EM2/GSK-795/GSK-458, EM2/Linsitinib/Dabrafenib, EM2/Linsitinib/Erlotinib, EM2/Linsitinib/Everolimus, EM2/Linsitinib/GSK-458, EM2/Linsitinib/Osimertinib, EM2/Linsitinib/Sorafenib, EM2/Linsitinib/Trametinib, EM2/Osimertinib/Alectinib, EM2/Osimertinib/Dabrafenib, EM2/Osimertinib/GSK-458, EM2/Osimertinib/Trametinib, EM2/Trametinib/GSK-795, EM2/Trametinib/Everolimus, EM2/Trametinib/GSK-458, EM3/Linsitinib/Sorafenib, EM3/Linsitinib/Dabrafenib, EM3/Cetuximab/Linsitinib, EM3/Dabrafenib/GSK-795, EM3/Erlotinib/GSK-795, EM3/Osimertinib/Dabrafenib, EM3/Osimertinib/GSK-795, EM3/Trametinib/Dabrafenib, EM3/Cetuximab/Dabrafenib, EM3/Cetuximab/GSK-795, EM3/Everolimus/Osimertinib, EM3/GSK-458/Cetuximab, EM3/Linsitinib/Osimertinib, EM3/Trametinib/Osimertinib, EM4/Trametinib/Dabrafenib, EM4/Sorafenib/Trametinib triple combinations where linsitinib can be replaced by any IGF-1R inhibitor, cetuximab can be replaced for any EGFR targeting biologic, osimertinib and erlotinib and be replaced by any EGFR inhibitor, dabrafenib can be replaced by any RAF inhibitor, trametinib can be replaced by any MEK inhibitor, GSK-795 can be replaced by any AKT inhibitor, GSK-458 can be replaced by any PI3K/mTOR dual specificity inhibitor, everolimus can be replaced by any mTOR inhibitor, and alectinib can be replaced by any ALK inhibitor.

In one aspect, the biomarker is the localization, and/or level, and/or state of a molecule, and/or organelle. In some aspects, the molecule being measured is a protein or a nucleic acid; the state of the molecule being measured is phosphorylation, acylation, alkylation, amidation, glypiation, glycation, glycosylation, ubiquitination, degradation product(s), truncation, mutation status, or binding of the molecule(s) to promoters; the localization of the molecule being measured is extracellular or cellular, wherein cellular localization includes intracellular, compartmentalized (e.g. Golgi, endoplasmic reticulum, lysosomal, endosomal, exosomal, mitochondrial, vacuole, cytosolic), nuclear or nucleoli, or membrane (e.g. plasma, nuclear, or organelle membranes) bound; and the state of the organelle being observed is nuclear vacuolation/nuclear autophagy, mitophagy. —is a biomarker.

In another aspect the biomarkers are the direct targets of an anti-cancer agent and/or modulator, wherein the targets include EGFR, ALK, VEGF A, VEGF B, VEGFR, VEGFR1, VEGFR2, VEGFR3, KIT, HER2, CDK4, CDK6, PARP, mTOR, PI3K, AKT1, AKT2, AKT3, n-RAS, k-RAS, c-RAS, a-RAF, b-RAF, c-RAF, MEK1, MEK2, ERK1, ERK2, PDGFRα or PDGFRβ, MET, RET, ROS1, PIGF, PTCH, Smoothened, RANKL, and B4GALNT1, PD-1, PD-L1 and PD-L2.

In other aspects, the biomarkers are signal transducer and activator of transcription proteins (STATs) STAT1, STAT3, or STAT5; receptor tyrosine kinases (RTKs; e.g. EGFR, HER2, EGFR3, EGFR4, IGF1R, MET, KIT, RET, PDGFR, VEGFR, ALK, BCR-ABL etc.); from the PI3K pathway (e.g. PTEN, PI3K, PDK1, AKT, mTOR, S6, p70 S6-Kinase, CREB, GSK3B, mTORC1 or mTORC2); from the MAPK pathway (e.g. Src, Fak, Ras, Raf, Mek, Erk, CREB, Sos-1, SHC, NFkB, cMyc, ELK-1, c-Fos or c-Jun); or are an immune checkpoint protein (e.g. PD-1, PD-L1 or PD-L2)

In another aspect, the biomarkers are markers of apoptosis and necroptosis (e.g. caspases 3, 6, 7, 8, 9, PARP, cytochrome c, and Bim, Bad, Bax, Bcl-2, Bcl-xL, and Mcl-1); markers of cell cycle (e.g. p27 Kip1, cyclin A, cyclin E, cyclin D, cyclin B, CDK1, CDK2, CDK4, CDK6, Cdc2, p16, p21, and p14); markers of proliferation (e.g. Ki67 and PCNA); markers of autophagy (e.g. LC3I, LC3II, LC3/LC3I/LC3II ratios, mTOR, AMPK, p62/SQSTM1, ATG5-12 complex, ATG13, Vps34, AMBRA-1 and UVRAG, GABA receptor-associated protein like 1, syntaxin-17, LAMP1, LAMP2, LAMP2B, p38, Beclin1, ATM, UNC-51-like kinase-1, -2, and -3). Predictive of epithelial mesenchymal transition (e.g. Snail, Slug, Twist, ZEB, and vimentin); are markers of necrosis (e.g. HMGB1); or cancer stem cell proteins (e.g. CD133, CD44, ABCG2, and ALDH1A1).

In some aspects, the molecule being measured is hypoxia inducible factor (HIF)-1α or HIF-2α.

In various aspects, the biomarker is Brdu/EdU incorporation and CFSE staining; the abundance of autophagosomes or autophagolysosomes, blebbing, or is a senescence assay (e.g. beta galactosidase assay).

In some aspects, a flow cytometry assay is used to measure necrotic, apoptotic and healthy cells (e.g. Hoechest and annexin V staining).

In one aspect, the biomarker is used to predict resistance is the baseline level of apoptosis markers.

In other aspects, the biomarker(s) are measured using immunoassays, multiplexed assays, PCR, transcription factor assays, nucleic acid or sequencing/mutation testing, cell survival and cell viability assays. In many aspects, the assay is an immunoassay (e.g. western blot, dot blot, ELISA, immunohistochemistry, immunocytochemistry, immunofluorescence).

In some aspects, the multiplexed assays is flow cytometry, microarrays, or bead-based such as Luminex multiplex assays.

In various aspects, the PCR, includes but is not limited to qPCR, RT-PCR, real-time PCR and endpoint PCR; the transcription factor identification assays is protein arrays, chromatin immunoprecipitation (CHIP) and CHIP-seq assays, DNA precipitation and DIP-seq assays, microsphere assays, DNase sensitivity or gel shift assays; the nucleic acid or sequencing/mutation testing includes all forms of sequencing DNA and RNA molecules, whole genome, exome, or specific genes only, including massively parallel signature sequencing (MPSS), 454 pyrosequencing, Illumina (Solexa) sequencing, SOLiD sequencing, Ion Torrent semiconductor sequencing, Heliscope single molecule sequencing, single molecule real-time (SMRT) sequencing, sequencing by hybridization, and sequencing with mass spectrometry; and the cell survival and cell viability assay, includes Hoechst 33342 and propidium iodide (HoPI) assay and MTT assay.

In many aspects, the biomarker or panel of biomarkers is used to predict the likelihood of response, and the biomarker is selected from the group consisting of EGFR, ALK, VEGF A, VEGF B, VEGFR, VEGFR1, VEGFR2, VEGFR3, KIT, HER2, CDK4, CDK6, PARP, cytochrome c, mTOR, PI3K, AKT1, AKT2, AKT3, n-RAS, k-RAS, c-RAS, a-RAF, b-RAF, c-RAF, MEK1, MEK2, ERK1, ERK2, PDGFRα or PDGFRβ, MET, RET, ROS1, PIGF, PTCH, Smoothened, RANKL, and B4GALNT1, PD-1, PD-L1, PD-L2, STAT1, STAT3, STAT5, HER2, EGFR3, EGFR4, IGF1R, MET, KIT, RET, PDGFR, VEGFR, ALK, BCR-ABL, PTEN, PDK1, S6, p70 S6-Kinase, CREB, GSK3B, mTORC1 and mTORC2, Src, Fak, Ras, Raf, Mek, Erk, CREB, Sos-1, SHC, NFkB, cMyc, ELK-1, c-Fos and c-Jun, caspases 3, 6, 7, 8, 9, PARP, cytochrome c, Bim, Bad, Bax, Bcl-2, Bcl-xL, and Mcl-1, p27 Kip1, cyclin A, cyclin E, cyclin D, cyclin B, CDK1, CDK2, CDK4, CDK6, Cdc2, p16, p21, p14, Ki67, PCNA, LC3I, LC3II, LC3/LC3II/LC3II ratios, AMPK, p62/SQSTM1, ATG5-12 complex, ATG13, Vps34, AMBRA-1 and UVRAG, GABA receptor-associated protein like 1, syntaxin-17, LAMP1, LAMP2, LAMP2B, p38, Beclin1, ATM, UNC-51-like kinase-1, -2, and -3, Snail, Slug, Twist, ZEB, vimentin, vinculin, HMGB1, CD133, CD44, ABCG2, ALDH1A1, Hif-1a, Hif2a, Brdu/EdU incorporation and CFSE staining, autophagosomes, autophagolysosomes, beta galactosidase, blebbing, Hoechest staining, annexin V staining, propidium iodide staining, WST8/MTT type assays, patient race, gender and age or age-related biomarkers such as follicle-stimulating hormone, biopsy type, tumor stage, tumor type and/or histological categorization including cell cycle status, cell type and differentiation status of the sample.

In one aspect, the expression levels of biomarkers in a specimen are compared with a non-responsive group and a responsive group of samples.

In various aspects, the expression level of a biomarker is normalized against a normalization biomarker, selected from the group consisting of actin, GAPDH, vinculin, tubulin, and histones. As used herein, the term “normalization biomarker” refer to any biomarker that can be used as a reference in a given assay, in order to allow proper quantification or the biomarker of interest, by comparing said expression levels of a biomarker of interest to the expression level of the normalization biomarker. Normalization biomarkers, or reference biomarkers are well known in the art, and various reference biomarkers are available; the most crucial aspect being that the reference gene must be stable. Non-limiting examples of normalization biomarkers include actin, GAPDH, vinculin, tubulin, a histone, ubiquitin, 18S and the like.

In other aspects, the comparison is performed by a software classification algorithm. In many aspects, the levels of the biomarkers are evaluated by applying a statistical method such as receiver operating characteristic (ROC) curve cut point analysis, regression analysis, discriminant analysis, classification tree analysis, random forests, support vector machine, OneR, kNN and heuristic naive Bayes analysis, neural nets and variants thereof.

In an additional aspect, the biological sample/cancer subject is treated in a manner to alter conditions such as proteins, carbohydrates, lipids, molecules found within fetal bovine serum, growth factors, hypoxia, oxidative stress, and/or physical exercise/stress.

As used herein “fetal bovine serum” or “FBS” refers to serum collected from the blood drawn of a bovine fetus via a closed system of collection. FBS is the most widely used serum-supplement for the in vitro cell culture of eukaryotic cells, mainly because it has a very low level of antibodies and contains more growth factors, allowing for versatility in many different cell culture applications. The globular protein, bovine serum albumin (BSA), is a major component of fetal bovine serum amongst the rich variety of proteins present in fetal bovine serum. As FBS is not a fully defined media component, and as such may vary in composition between batches.

Presented below are examples discussing the effects of combining modulator with anti-cancer therapy to enhance the efficacy of the treatment, contemplated for the discussed applications. The following examples are provided to further illustrate the embodiments of the present invention, but are not intended to limit the scope of the invention. While they are typical of those that might be used, other procedures, methodologies, or techniques known to those skilled in the art may alternatively be used.

### EXAMPLES

### Example 1

### Materials and Methods

Molecularly Targeted Agents (MTAs) and Enhancer Molecules (EMs)

Erlotinib hydrochloride was purchased from Selleck Chemicals (Catalog #51023). Recombinant human TNF-α was purchased from BD Biosciences (CA, USA).

Cell Culture:

Cell lines were obtained from the American Type Culture Collection or Sigma and maintained in manufacturer-recommended conditions in a humidified incubator at 37° C. with 5% carbon dioxide. Sub-cultivation and harvesting of all cell lines was performed with Acctuase (GIBCO, Life Technologies, Inc.) at 37 C for 5-10 min.

Antibodies:

pERK, pAKT, pGSK, PD-L1 were purchased from Cell Signaling. Snail and Bcl2 antibodies were purchased from Abcam (ab4224731) and Invitrogen (PAS-11379), respectively.

MTT Assay:

Cells (10⁴ cells/well) were grown in 96-well plates and exposed for 72 hours to different doses of erlotinib, alone or in combination with EM2. The percentage of cell survival was determined using the 3-(4, 5-dimethylthiazol-2-yl)-2, 5-diphenyltetrazolium bromide (MTT). Single drug alone and vehicle were kept as controls.

WST-8 Protocol

Day 1: cells were harvested cells from flask (4 mL accutase for T175 for 3-5 minutes), and spin down for 5 mins at 1200 rpm. The supernatant was poured off, and the pellet gently resuspended in 1 mL media. Countess counts were performed, and the cells were diluted in media (10% FBS). With multi-channel pipette, 2500 cells were seeded in 100 uL of media (10% FBS) in each well of 96 well plate. The cells were let to adhere and grow for 24 hours.
Day 2: serial drug dilutions of MTAs+/−EMs at 5× of concentrations (as done above) were performed in media (10% FBS). 20 uL of media form each well of the 96 well plate (80 uL media left in wells) was removed, and 20 uL of appropriate 5× drug dilutions was added to each respective well (for a 1× conc). The cells were incubated with drug for 72 hours in 37C incubator.
Day 5: 10 uL of WST-8 reagent was added to each well of the 96 well plate and mixed gently. the plates were incubated up to 4 hours at 37C in dark, and absorbance readings were taken at 450 nM and 600 nM wavelengths at 1, 2, 3 and 4h, until control absorbance levels were 1.5-2.0).

Immunocytochemistry (ICC) Staining:

Cells at the seeding density of 10,000 cells per well were plated in 8 chamber slides in complete RPMI culture medium for overnight. After 24h cells were treated with RPMI media without the serum along with selected dose of erlotinib+EM2 and kept for additional 72 hours in incubation condition set at 37° C. in a humidified atmosphere of 95% air and 5% CO₂. The chamber slides were rinsed with PBS before performing ICC staining. An Ambay kit (Catalog #10011A) purchased from Ambay Immune Sensors and Controls, LLC (AISC) (MD, USA) was used to perform ICC staining as per the kit instructions and using poly-HRP from Biogenix (CA, USA) was used to enable signal amplification and detection of specific bound antibody targets. A respective isotype antibody control was be kept for each biomarker. Counterstaining was performed before mounting and analysis.

HoPI Assay

A double stain of Hoechst 33342 and propidium iodide allows for the determination of cell viability with high specificity and low background noise, as compared to trypan blue staining. Hoechst 33342 is a cell permeable blue fluorescent nuclear stain which will stain all cell types. Propidium iodide is a cell impermeable red fluorescent nuclear stain that will only bind to the nuclei of cells with compromised membranes (dead cells). The staining can be performed rapidly on live cell suspensions within 15 minutes. 25 uL of 4× Hoechst 33342 Solution (40 ug/mL) and 25 uL of 4×Propidium Iodide Solution are added to 50 uL of cell suspension. The suspension was gently mixed 10 times and 50 uL was immediately transferred to a microfluidic cell counting chip. A 365 nm LED was used to excite both Hoechst 33342 and propidium iodide. Images of the cells were captured using a 10× objective lens on an Olympus microscope using a Canon EOS 6D camera using an ISO of 800 and an exposure of 2 seconds; both JPEG and RAW images were saved for analysis. The blue channel was used to measure the total number of cells and the red channel was used to measure the number of dead cells. Cell counting was performed automatically by the Image Pro software (Media Cybernetics, Inc).

Western Assay

Cells were grown on 10 cm2 plates and treated as described for the individual experiment. The supernatants were collected to get apoptotic bodies and extracted cells by accutase. Washed thoroughly and lysed total cell mix by using 1× lysis buffer (Biorad) with inhibitors. Lysate protein were separated using 4-12% or 10% SDS-PAGE gel and were transferred to PVDF membrane using Turbo blot (BioRad) as per the instructions. Membranes were blocked with 5% milk in TBS-Tween20 (0.1%) for 1 h at room temperature. Incubated with primary antibody p27 (1:4000), Ki67 (1:2000) overnight at 4-degree C. Next day incubated further 1 h at room temperature. Washed thrice in TBST buffer and incubated for 1 h at room temperature with respective HRP conjugated secondary antibody. Developed using clarity Western ECL substrate (BioRad) and exposed to x-ray films at various time to detect the proteins.

### Example 2

### Assessment of Interaction Between EM1 with MTAS in NSCLC, HNSCC, Melanoma, and CRC

We have generated data categorizing NSCLC, liver, renal colorectal, breast cancer, melanoma and HNSCC cell lines, xenografts, PDX, and clinical samples into sensitive and resistant groups for several therapies targeting EGFR and other RTKs, and the MAPK and PI3K pathways. During this work, we screened molecules to overcome acquired resistance. This led to the identification of several enhancer molecules (EMs). We have generated data, with preliminary data shown for EM-1-4, showing that these EMs can sensitize cancer cells to several therapies, including other EGFRi, RTKi, as well as ALKi, MEKi, AKTi, and others. The combinations can lead to decreased cell proliferation, cell cycle arrest, senescence, autophagy changes and induction of cell death-apoptotic, necrotic or autophagic cell death, as evidenced using various assays. Additionally, we have identified biomarkers to predict response to the combination treatments which can be optionally used to select patients into non-responders and responders.

| Enhancer | |
|---|---|
| Molecule | Identity/Example alternative names |
| EM1 | Interferon gamma, IFNg, IFNγ, |
| EM2 | Tumor necrosis factor alpha, TNFa, TNFα, |
| EM3 | Transforming growth factor beta, TGF beta, TGFβ |
| EM4 | Interleukin 6, IL-6 |

We used a panel of cell lines that included NSCLC, HNSCC, melanoma, and CRC. These cell lines are a good model system harboring similar (epi)genetic changes found in patients' tumors. We used the WST8 assay to determine the in vitro sensitivity to various MTAs with and without EM1. We have generated data demonstrating no, minimal and strong response enhancement.

### Example 3

### Assessment of Interaction Between EM2 with MTAS in NSCLC, HNSCC, Melanoma, and CRC

We used a panel of cell lines that included NSCLC, HNSCC, melanoma, and CRC. These cell lines are a good model system harboring similar (epi)genetic changes found in patients' tumors. We used the WST8 assay to determine the in vitro sensitivity to various MTAs with and without EM2. We have generated data demonstrating no, minimal and strong response enhancement.

### Example 4

### Assessment of Interaction Between EM3 with MTAS in NSCLC, HNSCC, Melanoma, and CRC

We used a panel of cell lines that included NSCLC, HNSCC, melanoma, and CRC. These cell lines are a good model system harboring similar (epi)genetic changes found in patients' tumors. We used the WST8 assay to determine the in vitro sensitivity to various MTAs with and without EM3. We have generated data demonstrating no, minimal and strong response enhancement.

### Example 5

### Assessment of Interaction Between EM4 with MTAS in NSCLC, HNSCC, Melanoma, and CRC

We used a panel of cell lines that included NSCLC, HNSCC, melanoma, and CRC. These cell lines are a good model system harboring similar (epi)genetic changes found in patients' tumors. We used the WST8 assay to determine the in vitro sensitivity to various MTAs with and without EM4. We have generated data demonstrating no, minimal and strong response enhancement.

### Example 6

### Assessment of EM2 Enhancement of Response to Erlotinib in NSCLC

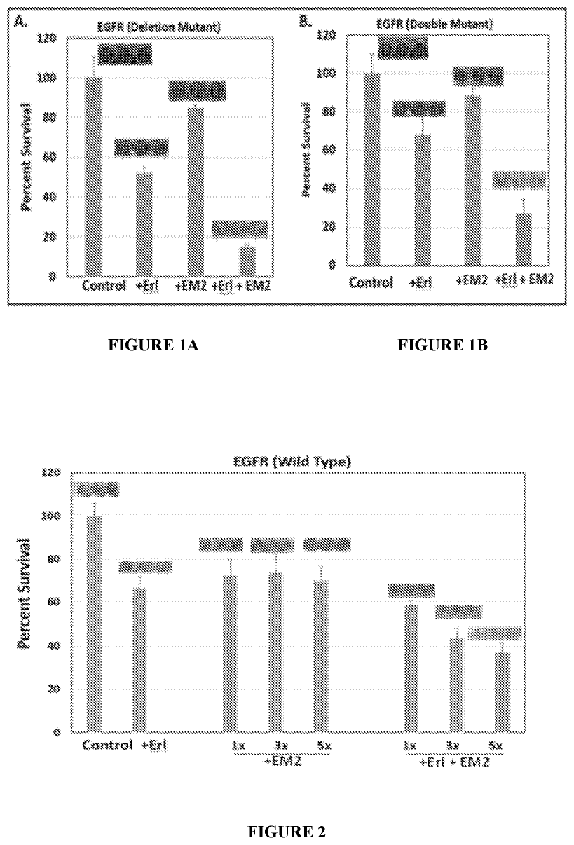

The effect of EM2 on cell response to Erlotinib was evaluated in various cancer cell lines, presenting different EGFR status; the impact on cell survival was evaluated by MTT assay. As illustrated in FIGS. 1 and 2, when 10 ng/ml of EM2 was combined with the EGFR inhibitor erlotinib (Erl) there was an increased sensitivity to Erl in an EGFR deletion mutant line HCC827 (see FIG. 1A). Erl-resistant EGFR-double mutant line H1975 were resistant to erlotinib (as see with the absence of effect of a 14 μM treatment), but an enhanced response was observed, corresponding to the reversion of the erlotinib resistance, upon treatment with EM2 (see FIG. 1B). Additionally, Erl-resistant EGFR WT cancer cells were shown to be sensitized to Erl by EM2 (see FIG. 2). These data suggested that our erlotinib+EM2 combination improved therapeutic efficacy for NSCLC patients irrespective of their EGFR mutation status.

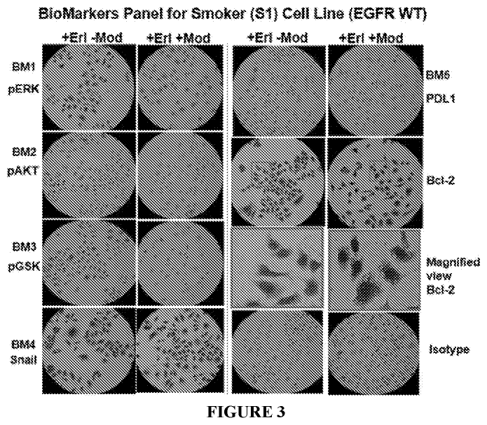

The monitoring of the changes in specific biomarkers from one of our diagnostic panels (BM1-BMS; p-Erk, p-Akt, p-GSK, Snail, PDL1, and Bcl2; in the presence of erlotinib (Erl) and EM2, indicated the involvement of pERK and pAKT in overcoming erlotinib resistance in WT EGFR smoker samples (see FIG. 3). The erlotinib+EM2 combination also promoted cell apoptosis which can be observed by changes in nuclear staining of Bcl2.

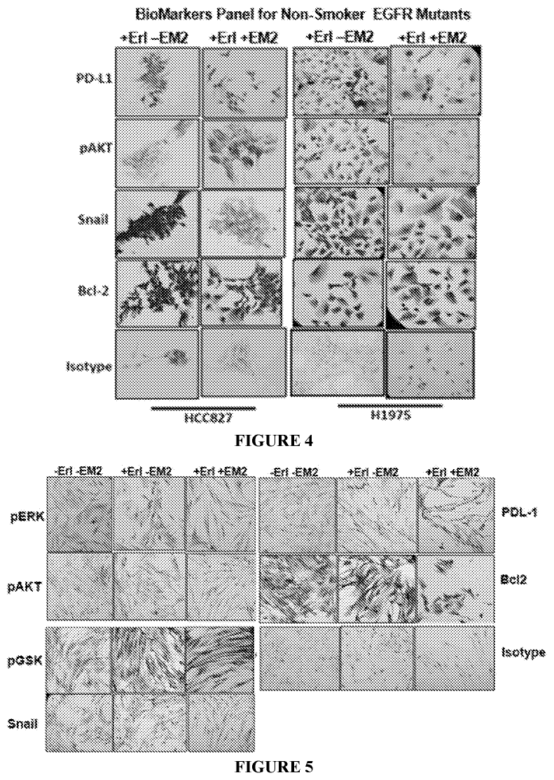

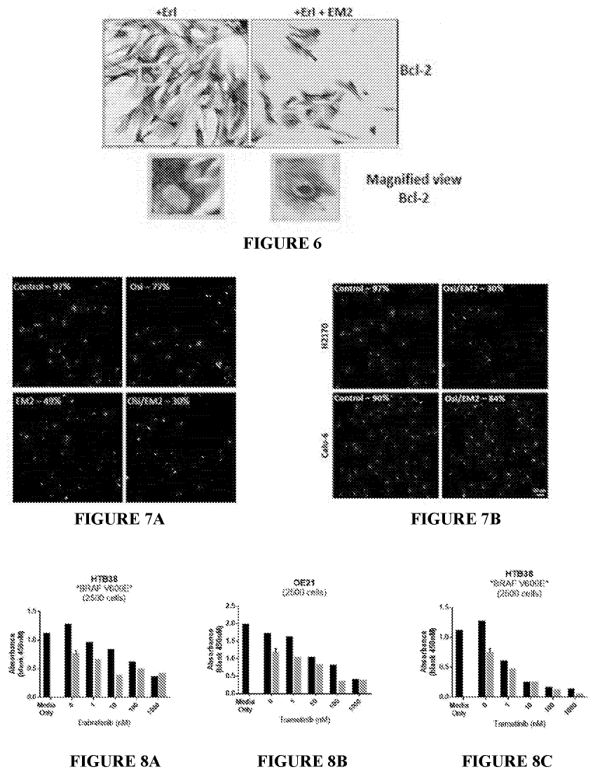

This panel of proximal and distal biomarkers can predict response to the combination therapies. Similar response profiles have been seen in other samples, including other cell lines (FIG. 4.) and a clinical sample (FIG. 5). The clinical sample was from patient who smoked and had a WT EGFR tumor. The erlotinib+EM2 combination promoted cell apoptosis, as demonstrated by changes in nuclear staining of Bcl2 as well as nuclear vacuolization (FIG. 6). Snail is an EMT marker that leads to emergence of cancer stem cells (CSC). This biomarker response panel and our other panels will assist in determination of both response and mechanism of action. For example, studying caspases 8, 3 and 7 gives insight and affirmation for cancer cell commitment to cell death. Examining p-STAT and NFkB covers the determination for PD-L1 expression, EMT and cell survival possibilities against the given treatments. Examining levels of other CSC biomarkers including CD44, CD133, ALDH1A and ABCG2 will help in understanding reversal of drug resistance & diminishing the possibility of relapse. EMT biomarkers such as TWIST, ZEB and vimentin, will help elucidate resistance to drug treatments, radiation etc.

### Example 7

### HoPI Data

As illustrated in FIGS. 7A and 7B, using a double stain of Hoechst 33342 and propidium iodide (HoPI assay), an increase in cell death was confirmed when the cells were treated with Osimertinib in combination with EM2.

### Example 8

### Material and Method to Measure Enhancement of Anti-Cancer/Anti-Growth Responses to Targeted Therapies Using Enhancer Molecules

To assess the enhancement effects of enhancer molecules (EM) to cell response to targeted therapy, various cell lines were used. The cells were treated with enhancer molecules, and biomarker levels of expression along with cell survival were studied in the cells treated with various targeted therapies.

The non-small cell lung cancer (NSCLC) cell lines Calu-6 (HTB-56), A427 (HTB-53), NCI-H2170 (CRL-5928), NCI-H1838 (CRL-5899), NCI-H460 (HTB-177), NCI-H1299 (CRL-5803), NCI-H292 (CRL-1848), NCI-H358 (CRL-5807), HCC827 (CRL-2868), NCI-H2228 (CRL-5935), NCI-H226 (CRL-5826), were purchased from American Type Culture Collection (ATCC), while PC-9 (#90071810) was purchased from MilliporeSigma. The mesothelioma cell line SK-MES-1 (HTB-58) was purchased from ATCC. The Head and neck squamous cell carcinoma (HNSCC) cell lines CAL27 (CRL-2095) and A-253 (HTB-41) were purchased from ATCC, while OE21 (#96062201), KYSE-70 (#94072012), BICR-16 (#06031001) and PE/CA-PJ15 (#96121230) were purchased from MilliporeSigma and Ca9-22 (JCRB-XenoTech #0625) was purchased from Sekisui XenoTech (Kansas City, Kans., USA). Melanoma cell lines SK-MEL-28 (HTB-72), SK-MEL-2 (HTB-68), RPMI-7951 (HTB-66), A7 (CRL-2500), colorectal cancer (CRC) cell lines HT-29 (HTB-38), HCT 116 (CCL-247), pancreatic cancer cell lines BxPC-3 (CRL-1687), HPAF-II (CRL-1997), kidney cancer cell line A-498 (HTB-44)) were purchased from ATCC. All cell lines were cultured according to the manufacturers' instructions in appropriate culture media. Culture medias RPMI-1640 medium (Cat. #30-2001), DMEM (Cat. #30-2002), EMEM (Cat. #30-2003), F-12K (Cat. #30-2004, IMDM (Cat. #30-2005), DMEM:F12 (Cat. #30-2006), McCoy's 5A (Cat. #30-2007) and L-15 (Cat. #30-2008) were purchased from ATCC. All cells were cultured at 37° C. and a humid atmosphere of 5% CO₂ in medium supplemented with 10% fetal bovine serum (FBS), a penicillin-streptomycin solution of 100 units/mL penicillin and 100 μg/mL streptomycin and 1 mM L-glutamine.

Erlotinib (OSI-744; Selleck Cat #S1023), Osimertinib (AZD9291; Selleck Cat #S7297), Dabrafenib (GSK2118436; Selleck Cat #S2807), Trametinib (GSK1120212; Selleck Cat #S2673), Linsitinib (OSI-906; Selleck Cat #S1091), Alectinib (CH5424802; Selleck Cat #S2762), Everolimus (RAD001; Selleck Cat #S1120), Uprosertib (GSK-795; GSK2141795; Selleck Cat #S7492), Omipalisib (GSK-458; GSK2126458; Selleck Cat #S2658) and Sorafenib (BAY 43-9006; Selleck Cat #S7397) were purchased from Selleck Chemicals. Spermidine (S2626), Metformin hydrochloride (PHR1084) and D-(+)-Trehalose dihydrate (T9531) were purchased from MilliporeSigma (St. Louis, Mo., USA). Enhancer molecules (EM) were purchased from MilliporeSigma, Cell Signaling Technologies (CST) and BD-BioSciences; EM1 is interferon-gamma (IFN-γ; IFNg; Sigma #11040596001), EM2 is tumor necrosis factor-alpha (TNF-α; TNFa; BD-BioSciences #554618), EM3 is transforming growth factor-beta-1 (TGF-β; TGFb; Sigma #T7039), EM4 is interleukin 6 (IL-6; Sigma #SRP3096), EM5 is interleukin 17 (IL-17; Sigma #SRP3080), EM6 is tumor necrosis factor-beta (TNF-β; TNFb; Sigma #T7799), EM7 is interferon-alpha-1 (IFN-α; IFNa; Sigma #SRP4596), EM8 is interleukin 10 (IL-10; Sigma #SRP3071), EM9 is interleukin 12 (IL-12; Sigma #SRP3073), EM10 is granulocyte-macrophage colony-stimulating factor (GM-CSF; CST #8922SF), EM11 is interleukin 1-alpha (IL-1α; IL1a; Sigma #12778) and EM12 is interleukin 2 (IL-2; Sigma #SRP6170).

The following antibodies used for immunoblotting were purchased from Cell Signaling Technologies (CST), Thermo Fisher Scientific and Bio-Rad Laboratories and used according to the manufacturer's instructions: phospho-AKT (Ser473; CST, Cat #4060), phospho-ERK (phospho-p44/42 MAPK; Thr202/Tyr204; CST, Cat #4376), GAPDH-HRP (CST, Cat #3683), LC3-A/B (CST, Cat #4108), p62/SQSTM1 (CST, Cat #5114), PARP (CST, Cat #9532), Ki-67 (SP6; Thermo Fisher, Cat #MA5-14520), Goat anti-mouse IgG-HRP (Bio-Rad, Cat #1706516) and Goat anti-rabbit IgG-HRP (Bio-Rad, Cat #1706515).

WST-8 Assay: MTA and EM effects on cell viability were measured by Cell Counting Kit8 (WST-8) colorimetric assay using water-soluble tetrazolium salt WST-8 reagent (Vita Scientific Cat. #DBOC00128). For the assay, cells were seeded in 96-well plates at 2,500 cells per well, in 100 μL of 10% FBS-containing media. After 24 hours, a 1 nM to 10 μM range of MTA and/or 1 ng/mL to 1 mg/mL range of EM doses was added and incubated for 72 hours. After treatment, a 10% volume of WST-8 reagent was added to each well and cells were incubated at 37° C. for up to 4 hours. Solution absorbance was measured at wavelengths of 450 nm and 600 nm at 1-, 2-, 3-, and 4-hour's incubation. Percentage viability of drug-treated cells was calculated relative to untreated control cells. Results were confirmed by three independent experiments.

MTA and EM effects on cell viability were also measured by MTT colorimetric dye reduction assay using MTT reagent (thiazolyl blue formazan; MilliporeSigma Cat. #M2003). For the assay, cells were seeded in 96-well plates at 3 concentrations: 25,000-, 10,000-, and 5,000 cell per well in 200 μL of 10% FBS-containing media. After 24 hours, a 1 nM to 1004 range of MTA and/or 1 ng/mL to 1 mg/mL range of EM doses was added and incubated for 72 hours. After treatment, drug media was removed, wells were washed twice with PBS, and 50 μL of 500 μg/mL MTT reagent was added to each well. Cells were then incubated at 37° C. for 2 hours, formazan product was solubilized in 200 μL DMSO, and absorbance was measured at a wavelength of 570 nm. Percentage viability of drug-treated cells was calculated relative to untreated control cells. Results were confirmed by three independent experiments.

MTA and EM effects on protein biomarker expression were measured by western blotting. For the assay cell lines (Ca9-22, PE/CA-PJ15, HTB-38, HPAF-II, CAL27, BICR-16, A-253, Calu-6, PC-9, H1838, RPMI-7951 and A7) were cultured and treated with doses of MTAs alone, EMs alone and in combination and then lysed for protein analysis. This method can readily be conducted under adherent or suspension conditions, manually or on automated instruments, such as SnapPath. A total of 0.3×10⁶ cells per well were seeded in 6 well plates with 10% FBS-containing media and incubated for 24 hours. Cells were then treated with MTA and/or EM concentrations for 24 hours. For total protein extraction, briefly, each sample media was collected, and wells were washed with HMS, and the wash was combined with its respective media collection. Cells were then detached using Accutase solution and again collected in its respective sample media and another HMS well wash and collection was performed. All cell solutions were centrifuged, washed with ice-cold PBS, and centrifuged again. Cell pellets were lysed in 250 μL of ice-cold 1× lysis buffer with protease and phosphatase inhibitor cocktails and incubated for 10 minutes on ice. Lysates were clarified with a centrifugation (14,000 rpm) for 10 minutes at 4° C. Cell lysates were quantified using Bio-Rad DC Protein Assay (Bio-Rad Cat. #5000116).

For all western blots, an equal amount of protein (3-5 μg) was loaded into SDS-polyacrylamide gels (Mini-Protean TGX Precast Gel; Bio-Rad, e.g. 7.5%, Cat. #4561024; 10%, Cat. #4561035; 12%, Cat. #4561045; or 4-15%, Cat. #4561085). Proteins were separated at 215V for at least 30 minutes in 1× Tris/Glycine/SDS buffer (Bio-Rad Cat. #1610772) and transferred to PVDF membranes using a Trans-Blot Turbo (Bio-Rad Cat. #1704155) for 7 minutes in transfer buffer (Trans-Blot Turbo Transfer Kit, Bio-Rad Cat. #1704273). Blots were blocked at room temperature for 1 hour with 5% non-fat dry milk in tris-buffered saline with Tween 20 (TBST; 15 mM Tris-HCl, pH 7.5, 200mMNaCl and 0.1% Tween 20). Membranes were incubated with primary antibodies at 4° C. overnight and at room temperature for 1 hour the next day; all primary antibodies were diluted following manufacturer's specifications in TBST with either 5% BSA or 5% non-fat dry milk. Membranes were washed in TBST for 5 minutes, three times, and incubated with HRP-conjugated secondary antibodies diluted 1:5000 in TBST with 5% non-fat dry milk at room temperature for 1 hour (anti-rabbit IgG, Bio-Rad Cat. #1706515; or anti-mouse IgG, Bio-Rad Cat. #1706516). Membranes were then washed in TBST for 10 minutes, three times, and target proteins were identified using Clarity Western ECL Substrate (Bio-Rad Cat. #1705061) on autoradiography film (GenHunter Cat. #B581, Nashville, Tenn., USA). Other assay types, such as Luminex multiplexed immunoassays, ICC, IHC, flow cytometry, etc. can be used readily.

MTA and EM effects on apoptosis were measured by Hoechst-Propidium Iodide staining (HoPI). For the assay, cells were seeded in 6-well plates at 400K cells per well, in 2 mL of 10% FBS-containing media and incubated for 24 hours. At 24 hours, a 1 nM to 1004 range of MTA and/or 1 ng/mL to 1 mg/mL range of EM doses was added and incubated for 24 hours. After 24 hours incubation respective cells' media was collected, added the 1× HBSS wash from the plate in the respective tube. Adherent cells were detached by treating with Accutase for 6 minutes and collected in respective tubes. All samples were centrifuged for 5 minutes at 1200 RPM. The cell pellets were resuspended in PBS and centrifuged again for 5 minutes at 1200 RPM. Cell pellets were resuspended in 200 uL of PBS and mixed properly to get single cell suspension. 50 uL of cell suspension was added into separate tubes having 10 uL of a 2× HOPI solution (20 ug/mL Hoechst 33342 and 20 ug/mL propidium iodide solution) to get a final concentration of Hoechst 33342 and propidium iodide to 0.2 ug/mL. Make up the volume to 100 uL with 40 uL of PBS. Samples were mixed thoroughly for even cell staining and the microfluidsic chip wells were each filled with 10 uL of sample. Chips were then incubated in the dark for 20 minutes. A 365 nm LED was used to excite both Hoechst 33342 and propidium iodide as they were visualize under a microscope. Cell images were captured using a 10× objective lens on an Olympus microscope using a Canon EOS 6D camera with an ISO of 800 and an exposure of 2 seconds; both JPEG and RAW images were saved for analysis. The blue channel was used to measure the total number of cells and the red channel was used to measure the number of dead cells. For image analysis, a copy of the image was created and converted directly into an 8-bit gray scale image. A threshold was placed on the image to create a binary mask; the threshold level was 20 intensity units out of 255 intensity units. Next, a particle analysis algorithm was applied to the binary mask and a set of cellular regions (between 100 to 800 cellular regions per image) was created. The original image was then split into red, green and blue channels. The area, mean intensity, standard deviation of the mean intensity, minimum intensity, maximum intensity and perimeter were measured for each cellular region in the red channel and also in the blue channel. The green channel was discarded. To discern between live and dead cells, the maximum intensity in the red channel was used with a threshold of 100 intensity units. Double plots were created using the intensity of the blue channel (X-axis) and the intensity of the red channel (Y-axis). The number of live cells was calculated based on the number of cells that had red channel intensity less than 100 intensity units.

### Example 9

### Enhancement of Anti-Growth Responses to Targeted Therapies Using Enhancer Molecules

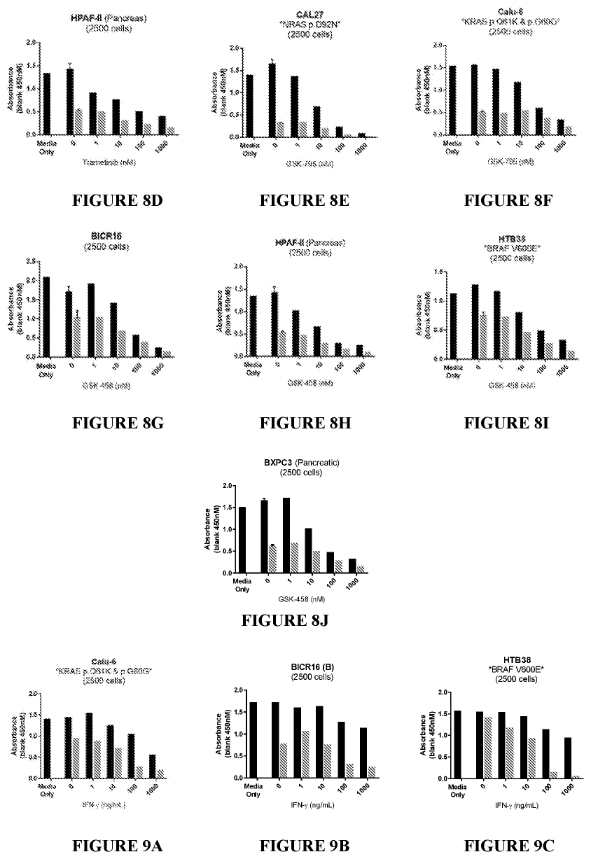

For studying the effects of various molecularly targeted agents (MTAs) and enhancer molecules (EMs) alone as well as in combination: HCC827, PC-9, A427, Calu-6, H2228, H358, H1299, H460, H292, H1838, H2170, and H226 (non-small cell lung cancer; NSCLC); and SK-MES-1 (mesothelioma), CAL27, OE21, KYSE-70, A-253, BICR-16, PE/CA-PJ15 and Ca9-22 (head and neck squamous cell carcinoma; HNSCC); SK-MEL-2, SK-MEL-28, RPMI-7951 and A7 (melanoma); HTB-38 and HCT116 (colorectal cancer; CRC); BxPC-3 and HPAF-II (pancreatic cancer); HEPG2 (hepatocellular carcinoma; HCC); A498 (renal cell carcinoma; RCC) cells were used. Other cell lines, including those from other cancer types, or cells collected from more complex sample types such as xenografts and clinical samples can be used. Ultimately, these combinations can be utilized in various clinical and pre-clinical samples and studies. Using the WST-8 cell viability assay, a MTA+EM-induced anti-cancer/anti-growth activity in multiple cell lines was discovered (FIGS. 8-15). Other viability assays, for example, MTT, HoPI staining, dye exclusion (e.g. trypan blue), can be used readily. The cell line panel showed varying responses to a range of doses for each MTA monotherapy, and through combinations with fixed sub-lethal doses of various EMs many MTA-resistant/minimally sensitive cell lines were subsequently turned into fully responsive ones. The cut-off value for classifying a “strong anti-cancer/anti-growth enhancement” was a response resulting in >70% reduction in cell survival compared to untreated control cells (100%), while showing greater reduction compared to both MTA and EM alone. In the following sections such cell line models, which were shown to be susceptible to “anti-cancer/anti-growth enhancement” when MTA monotherapies were combined with each respective EM, were identified.

Using the WST-8 assay, and as illustrated in FIGS. 8A-8J, IFN-γ showed moderate-to-strong anti-cancer/growth enhancement in combination with dabrafenib (BRAE inhibitor) in HTB-38 (BRAE V600E), with trametinib (MEK inhibitor) in HTB-38, OE21 and HPAF-II, with GSK-795 (pan-AKT inhibitor) in CAL27 (NRAS p.D92N) and Calu-6 (KRAS p.Q61K) and with GSK-458 (PI3K/mTOR inhibitor) in HTB-38, BICR-16, HPAF-II, and BxPC-3.

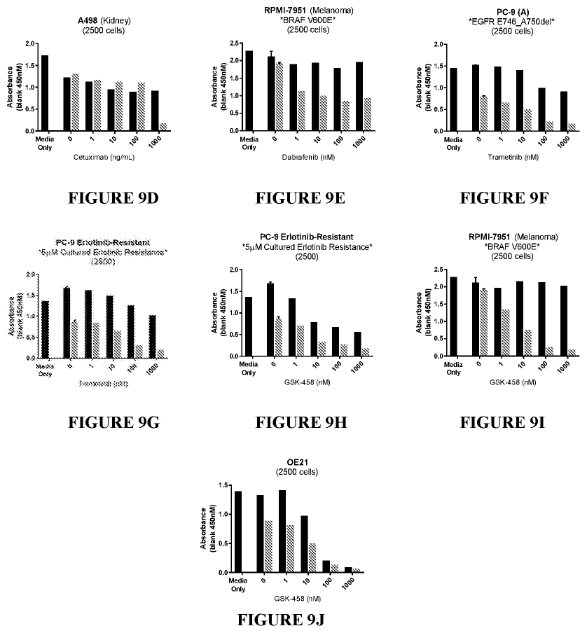

TNF-α (FIGS. 9A-9J) showed moderate-to-strong anti-cancer/growth enhancement in combination with IFN-γ in Calu-6, BICR-16 and HTB-38, with cetuximab (EGFR inhibitor) in A498, with dabrafenib in RPMI-7951 (BRAE V600E), with trametinib in PC-9 (EGFR E746_A750del) and erlotinib-resistant PC-9 and with GSK-458 in erlotinib-resistant PC-9, RPMI-7951 and OE21.

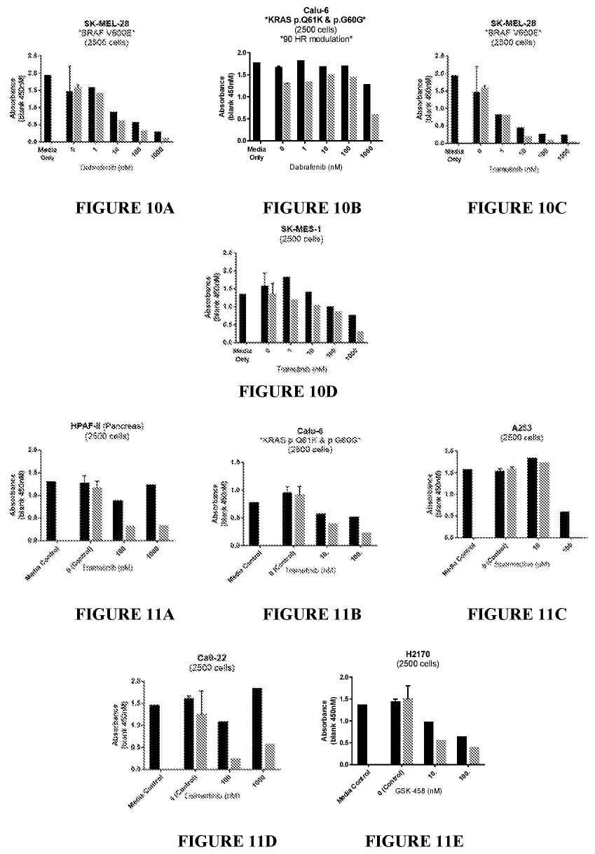

TGF-β (FIGS. 10A-10D) showed anti-cancer/growth enhancement in combination with dabrafenib in SK-MEL-28 (BRAE V600E) and Calu-6 and with trametinib in SK-MEL-28 and SK-MES-1.

IL-17 (FIGS. 11A-11E) showed moderate-to-strong anti-cancer/growth enhancement in combination with trametinib in Calu-6 and HPAF-II, with osimertinib (EGFR inhibitor) in Ca9-22, with GSK-458 in H2170 and with spermidine (autophagy inducer) in A-253.

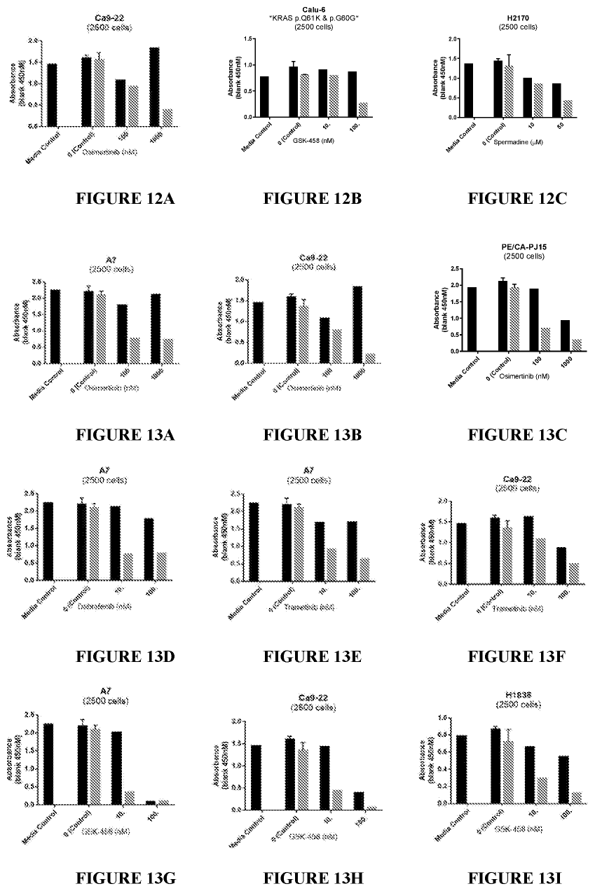

TNF-β (FIGS. 12A-12C) showed anti-cancer/growth enhancement in combination with osimertinib in Ca9-22, with GSK-458 in Calu-6 and with spermidine in H2170.

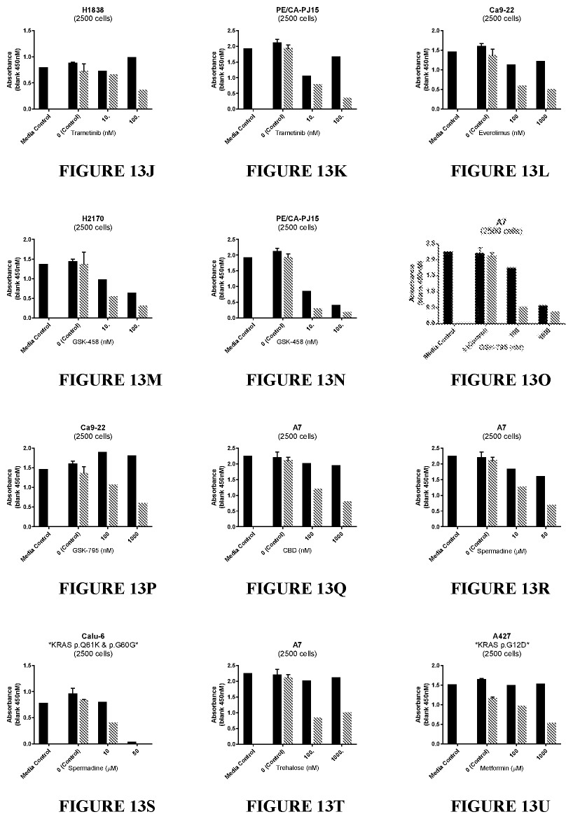

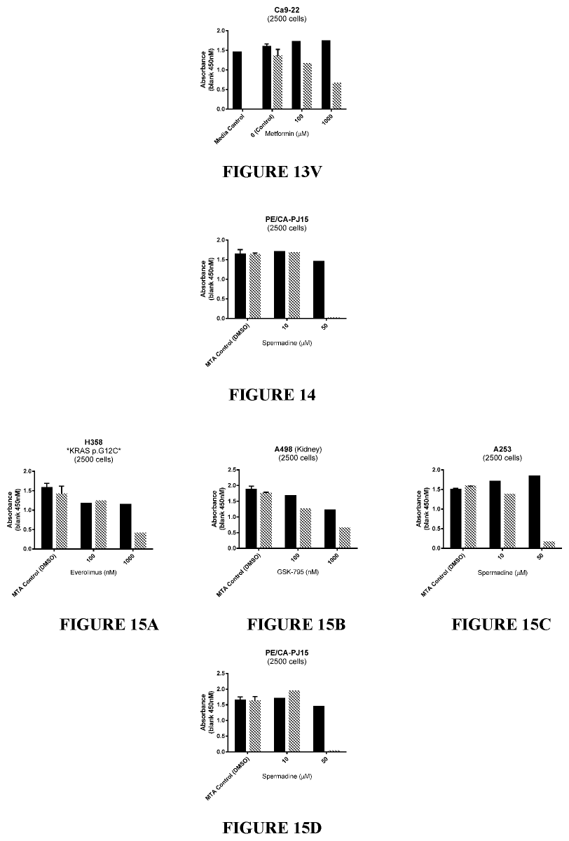

IFN-α (FIGS. 13A-13V) showed anti-cancer/growth enhancement in combination with dabrafenib in A7, with trametinib in A7, Ca9-22, H1838 and PE/CA-PJ15, with osimertinib in A7, Ca9-22 and PE/CA-PJ15, with GSK-458 in A7, Ca9-22, H1838, H2170 and PE/CA-PJ15, with GSK-795 in A7 and Ca9-22, with everolimus in Ca9-22, with spermidine in A7 and Calu-6, with CBD (phytocannabinoid factor) in A7, with trehalose (autophagy inducer) in A7 and with metformin (antihyperglycaemic agent) in Ca9-22 and A427.

IL-12 (FIG. 14) showed anti-cancer/growth enhancement in combination with spermidine in PE/CA-PJ15.

GM-CSF (FIGS. 15A-15D) showed anti-cancer/growth enhancement in combination with GSK-795 in A498, with everolimus in H358 (KRAS p.G12C) and with spermidine in A-253 and PE/CA-PJ15.

### Example 10

### Enhancement of Anti-Cancer Responses to Targeted Therapies Using Enhancer Molecules

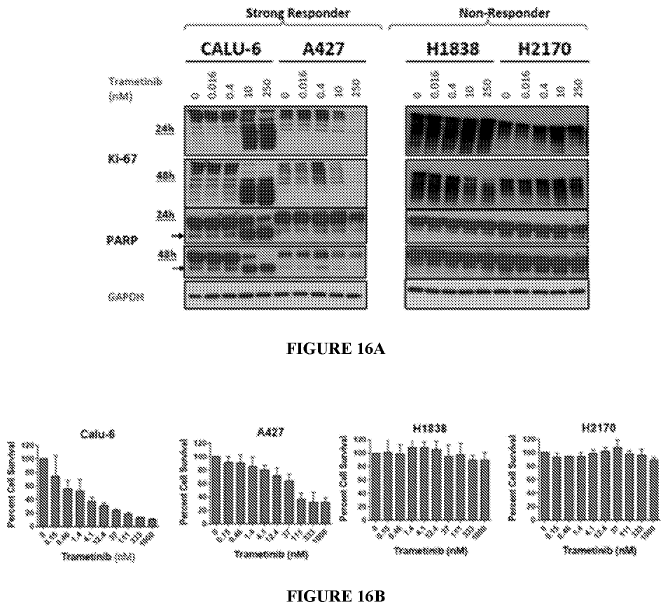

To determine the full range of physiological outcomes in response to the EMs in combination with MTAs and to identify predictive biomarkers of response, effects on cell proliferation and multiple types of cell death including apoptosis, necroptosis as well as autophagy were evaluated. These biomarkers can be used to identify each MTA+EM combination's mechanism of action for development of a diagnostic tool for every effective combination. First, MTA+EM combination effects on the Ki67 protein, an indicator of cell growth and proliferation in cancer, were assessed. The data obtained after treating NSCLC cells with trametinib alone revealed unique Ki67 expression pattern in trametinib-responsive cells (FIG. 16).

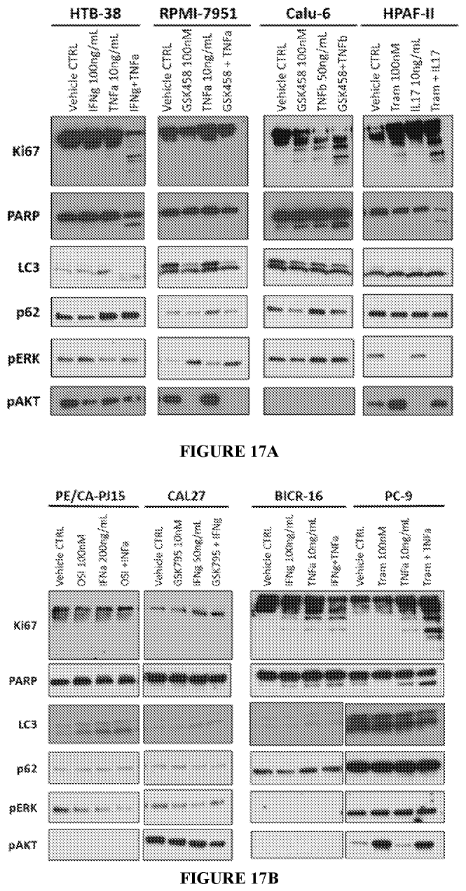

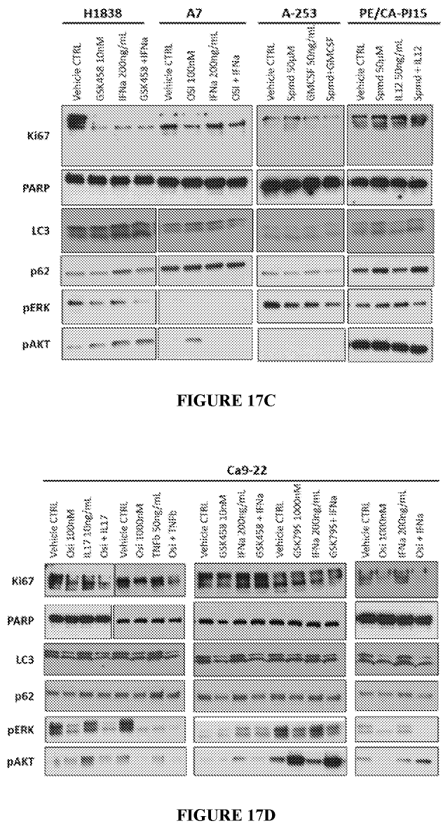

This phenomenon was further explored using other MTA and EM combinations and the unique degradation pattern of Ki67 was discovered in western blot as a co-indicator of apoptosis in effective combination treatments (FIG. 17). Earlier studies have used the presence or absence of Ki67 expression as a static biomarker for response in solid tumors but it has not been utilized as a dynamic marker exhibiting dose-responsive degradation patterns. Static Ki67 levels do not appear to be as predictive as the unique dynamic responses observed. A marked increase in the distinct degradation and/or fragmentation pattern of Ki67, occurring in combination treatments at 24 hours, was observed when compared to either therapy alone in CRC cell line HTB-38 treated with IFN-γ (100 ng/mL)+TNF-α (10 ng/mL), NSCLC cell lines Calu-6 treated with GSK-458 (100 nM)+TNF-β (50 ng/mL) and PC-9 with trametinib (100 nM)+TNF-α (10 ng/mL), HNSCC cell line BICR-16 treated with IFN-γ (100 ng/mL)+TNF-α (10 ng/mL), as well as in pancreatic cell line HPAF-II treated with trametinib (100 nM)+IL-17 (10 ng/mL). The data showed that other unique MTA+EM combinations can induce anti-cancer effects by an enhanced reduction in Ki67 expression, compared to both MTA and EM alone, which is further indication of an enhancement in anti-cancer mechanisms. These cases include HNSCC cell line A-253 treated with spermidine (50 μM)+GM-CSF (50 ng/mL) and HNSCC Ca9-22 cells treated with osimertinib (100 nM)+IL-17 (10 ng/mL).

The Ki67 predictive biomarker (distinct degradation pattern) data was complemented by monitoring for cleavage of the key apoptotic marker PARP, confirming an induction of apoptotic cell death in many combinations treated at 24 hours. In HTB-38 cells treated with IFN-γ (100 ng/mL)+TNF-α (10 ng/mL), Calu-6 cells treated with GSK-458 (100 nM)+TNF-β (50 ng/mL), HPAF-II cells treated with trametinib (100 nM)+IL-17 (10 ng/mL), PC-9 cells treated with trametinib (100 nM)+TNF-α (10 ng/mL) and BICR-16 cells treated with IFN-γ (100 ng/mL)+TNF-α (10 ng/mL). In these cases, we observed very strong PARP cleavage, compared to MTA and EM alone, indicating a clear enhancement of apoptotic mechanisms in these cell lines after combination treatments.

The distinct pattern of Ki67 degradation, coupled with enhanced cleavage of PARP in many cell lines treated with EM combinations indicated a clear enhancement of anti-cancer/pro-apoptotic effects when compared to either single therapy alone; this supports these Ki67 and PARP patterns as predictive biomarkers for the more beneficial combination therapies wherein enhancement of anti-cancer effects of PI3K and MAP Kinase inhibitors was observed with addition of sub-lethal doses of TNF-α, TNF-β, IFN-γ, or IL-17.

MTA+EM combination effects were further assessed on proximal biomarkers in the PI3K/AKT and MAP Kinase pathways (FIG. 17). Weak therapeutic responses, as well as mechanisms of resistance, to targeted therapies in cancer can be caused by suppression of negative feedback mechanisms and the induction of parallel or compensatory signaling pathways. The internal data with trametinib alone showed phospho-AKT (p-AKT) induction following treatment was a negative indicator of overall trametinib response. Therefore, AKT phosphorylation status could yield predictive biomarkers for response to our effective MTA+EM combinations. A strong reduction in p-AKT (Ser473) expression was observed in some EM combination treatments over 24 hours, when compared to either MTA or EM alone. These included CAL27 cells treated with GSK-795 (10 nM)+IFN-γ (50 ng/mL), HPAF-II cells treated with trametinib (100 nM)+IL-17 (10 ng/mL), PC-9 cells treated with trametinib (100 nM)+TNF-α (10 ng/mL) and Ca9-22 cells treated with osimertinib (100 nM)+IL-17 (10 ng/mL). In the cases of HPAF-II and PC-9, cells treated with trametinib alone showed significantly increased p-AKT, compared to control, which shows an induced survival mechanism against the MTA. However, this trametinib-induced p-AKT expression was subsequently inhibited by addition of EM (by IL-17 in HPAF-II and by TNF-α in PC-9). The combination with IL-17 or TNF-α reverses MTA-induced overexpression of p-AKT and inhibits a potential AKT-driven survival mechanism. These data show that EMs like IL-17 and TNF-α can be used as an effective anti-resistance enhancer molecules for MTAs which activate compensatory pathways in targeted therapy resistance development. These p-AKT responses can be combined with observed Ki67 and PARP responses. For example, the IL-17 and trametinib combination enhances Ki67 degradation, PARP cleavage and p-AKT down regulation in HPAF-II cells. Altogether, the data demonstrate that dynamic p-AKT changes, along with Ki67 and PARP, can be used to predict effective MAP Kinase-targeting MTAs+EM combinations.

### Example 11

### Enhancement of Autophagic or Necroptotic States in MTA-Resistant Cell Lines Using Enhancer Molecules

Most targeted therapeutic agents against cancer induce a cytostatic effect, as opposed to a strong cytotoxic effect which can result in clear cell death mechanisms like apoptosis. To explore this cytostatic nature of many therapies, potential autophagy induction was assessed in cases where cell growth was inhibited yet apoptosis activation was not evident. Autophagy is a flux state of cellular fate that can halt cell oncogenesis and ultimately lead to cell death in certain instances. To assess autophagic processes in our most effective anti-cancer/growth MTA+EM combinations, expression of autophagy-related proteins LC3 and p62 were examined (FIG. 17). Due to its common localization in autophagosome membranes, an increased expression of LC3II compared to LC3I correlates with and predicts an increase in autophagosome number and thus induced autophagic activity within cells. A marked increase in LC3II over LC3I expression was observed in some EM combination treatments over 24 hours, when compared to either MTA or EM therapy alone. These cases include Ca9-22 cells treated with GSK-458 (10 nM)+IFN-α (200 ng/mL), CAL27 cells treated with GSK-795 (10 nM)+IFN-γ (50 ng/mL), BICR-16 cells treated with IFN-γ (100 ng/mL)+TNF-α (10 ng/mL), as well as PE/CA-PJ15 cells treated with combinations of osimertinib (100 nM)+IFN-α (200 ng/mL) and spermidine (50 μM)+IL-12 (50 ng/mL). p62 also shares a key role in autophagy. s an autophagic receptor, p62 delivers substrates to autophagosomes for degradation during which it is degraded. A marked reduction in p62 expression would correlate with an increase in autophagic flux state, which can lead to autophagic cell death. Our data showed that CAL27 cells treated with GSK-795 (10 nM)+IFN-γ (50 ng/mL) had reduced p62 expression along with induction of LC3II, when compared to either therapy alone, showing enhancement of active autophagy flux state. Similarly, Ca9-22 cells treated with osimertinib (1000 nM)+IFN-α (200 ng/mL) and A-253 cells treated with spermidine (50 μM)+GM-CSF (50 ng/mL) showed a reduction in p62 along with LC3II upregulation in the MTA+EM combination compared to either alone. Contrary to these, HTB-38 cells treated with IFN-γ (100 ng/mL)+TNF-α (long/mL) and PE/CA-PJ15 cells treated with either spermidine (50 μM)+IL-12 (50 ng/mL) or osimertinib (100 nM)+IFN-α (200 ng/mL) increased expression of both LC3II and p62. These results demonstrate a block in autophagic flux or potential necroptosis induction. Blocking this MTA-induced autophagy may inhibit resistance development against the MTA.

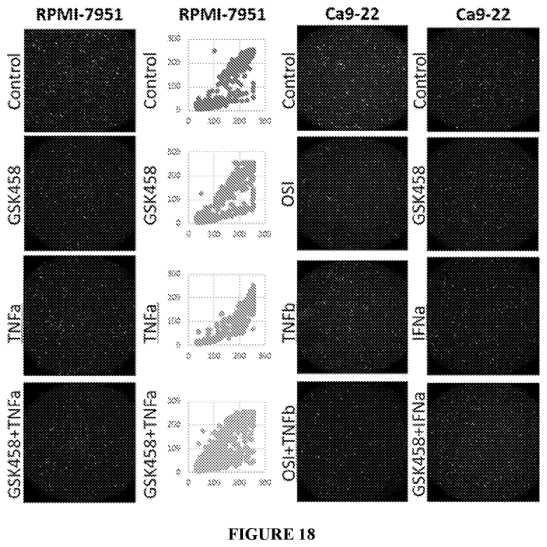

As way of example of live cell staining we used HoPI. RPMI-1975 had a 30% reduction and 8% reduction in live cells from GSK458 and TNFa treatment respectively. In combination, a 68% reduction in live cells was observed in RPMI-1975. The significant cell death with the combination treatment in RPMI-1975 can be the result of necrosis, necroptosis or autophagic cell death. This can also be observed from the images and the double plot dataset. In Ca-922 cell line, a 41% reduction and 21% reduction in the number of live cells was observed for osimertinib and TNFa respectively. In combination, a 67% reduction in the number of live cells was observed. This type of enhancement was not observed using Ca-922 treated with GSK458 and IFNa, with a 22% and 24% reduction in the number of live cells in GSK458 and IFNa alone treatment, and 26% reduction in combination treatment group (FIG. 18). These data show that the enhancement of the effect can be detected using HoPI staining, including images, double plots and the quantitative results and can be used to predict long term outcomes.

Therapeutically effective and novel combinations of MTAs with biological compounds or two different biological compounds against an array of cancer types were discovered in this work. Many MTA+EM combinations lead to unique anti-cancer outcomes. Some combinations inhibit proliferative growth and induce apoptotic death mechanisms, while other combinations cause growth retardation and/or cell death from autophagic or necroptotic mechanisms. Furthermore, other combinations block drug resistance mechanisms. A unique Ki67 degradation pattern which can predict therapeutic outcome was discovered. Collectively, dynamic responses of Ki67, PARP, p-AKT, LC3/p62 and HOPI responses can be used as predictive biomarkers for identifying effective MTA/EM therapeutic combinations either as standalone biomarkers or part of a multiplex panel.

### REFERENCES

1. American Lung Association Scientific and Medical Editorial Review Panel (2016). Lung Cancer Fact Sheet. American Lung Association. www.lung.org/lung-health-and-diseases/lung-disease-lookup/lung-cancer/resource-library/lung-cancer-fact-sheet. html
2. Soria J C et al., (2018). Osimertinib in Untreated EGFR-Mutated Advanced Non-Small-Cell Lung Cancer. N Engl J Med. 378: 113-125.
3. Domvri K, et al., (2013). Molecular Targeted Drugs and Biomarkers in NSCLC, the Evolving Role of Individualized Therapy. J. Cancer. 4: 736-754.
4. Lindeman N I et al. (2013). Molecular testing guideline for selection of lung cancer patients for EGFR and ALK tyrosine kinase inhibitors: guideline from the College of American Pathologists, International Association for the Study of Lung Cancer, and Association for Molecular Pathology. J Thorac Oncol. 8:823-859.
5. Polychronidou G and Papakotoulas P, (2013). Long-Term Treatment with Erlotinib for EGFR Wild-Type Non-Small Cell Lung Cancer: A Case Report. Case Rep Oncol. 6: 189-196.
6. American Cancer Society. Cancer Facts & FIGS. 2018. Atlanta: American Cancer Society; 2018.
7. Chien-Hua Tseng et al., (2017). EGFR mutation, smoking, and gender in advanced lung adenocarcinoma Oncotarget 58: 98384-98393.
8. Barbor M, (2017). A Decade of Lessons Learned From EGFR-Targeted Therapy. The ASCO Post. http://www.ascopost.com/issues/march-10-2017/a-decade-of-lessons-learned-from-egfr-targeted-therapy/9.
9. Wang S et al., (2017). EAI045: The fourth-generation EGFR inhibitor overcoming T790M and C797S resistance. Cancer Lett. 385: 51-54.
10. Nan X et al. (2017) EGFR TKI as first-line treatment for patients with advanced EGFR mutation-positive non-small-cell lung cancer. Oncotarget. 8:75712-75726.

Although the invention has been described with reference to the above examples, it will be understood that modifications and variations are encompassed within the spirit and scope of the invention. Accordingly, the invention is limited only by the following claims.

1. A method of enhancing the response of a biological sample/cancer subject to one or more anticancer therapies comprising:
a) contacting the sample/subject with one or more modulators prior to, during, simultaneously with, throughout, or following the anticancer therapies to alter the levels, state, or localization of key targets to increase the efficiency of treatment as monotherapy or combination therapy; and
b) optionally measuring the levels, state, or localization of key targets and/or related biomarkers in the biological sample/cancer subject to predict response to the therapy(ies) and modulator(s).

2. The method of claim 1 wherein the one or more anticancer therapies and/or the one or more modulators are administered by various means selected from the group consisting of intravenous, intraperitoneal, intra/transdermal, intratumoral, subcutaneous, inhalation, ocular, sublingual, epidural, vaginal, intranodal, transmucosal, and rectal routes.

3. The method of claim 1, wherein the cancer is colorectal, esophageal, stomach, lung, mesothelioma, prostate, uterine, breast, skin, endocrine, melanoma, urinary, pancreas, ovarian, cervical, head and neck, liver, bone, biliary tract, small intestine, hematopoietic/blood (myeloma, leukemia, and lymphoma), vaginal, testicular, anal, kidney, brain, eye cancer, leukemia, lymphoma, or soft tissue cancer, melanoma, mixed types, and metastases thereof.

4-6. (canceled)

7. The method of claim 1, wherein response and biomarkers are measured in vivo, ex vivo, or in vitro.

8. The method of claim 1, wherein the sample is processed for ex vivo or in vitro analysis using one or more manual methods and/or automated systems.

9. The method of claim 1, wherein the biological sample is a cancer cell, immune cell, stromal cell, or sub-population thereof.

10. The method of claim 1, wherein the biomarker is measured on tumor cells, stromal cells, immune cells or subpopulations thereof; wherein the tumor cells comprise cancer stem cells, wherein the cancer stem cells express one or more of CD133, CD44, ABCG2, and/or ALDH1A1; wherein the stromal cells are fibroblasts; and wherein the immune cells are selected from the group consisting of T cells, B cells, NK cells, dendritic cells, myeloid derived suppressor cells, macrophages, granulocytes, and mast cells.

11. The method of claim 1, wherein the biological sample is obtained by blood draw, fine needle aspiration, core biopsy, surgical excision, or other tumor sample acquisition method from a model organism or a subject/cancer patient.

12. The method of claim 1 wherein the modulator is a phenothiazine, a cytokine, a cannabinoid, a polyphenol, an autophagy modulator, a derivative thereof, a mutant thereof, a peptide thereof, a fragment thereof, an analog thereof or a mimetic thereof.

13. (canceled)

14. The method of claim 12, wherein the cytokine is interferon (IFN) gamma, tumor necrosis factor (TNF) alpha, tumor growth factor (TGF) beta, interleukin (IL) 6, or IL-17.

15. The method of claim 12, wherein the cannabinoid is synthetic THC-Delta-9 THC or cannabidiol (CBD).

16. (canceled)

17. The method of claim 12, wherein the autophagy modulator is metformin, melatonin, trehalose, spermidine, spermine, azithromycin, chloroquine, or chloramphenicol.

18. The method of claim 1 wherein the anticancer therapy is replaced by a modulator, wherein the modulator is a cytokine, a cannabinoid, a polyphenol an autophagy modulator, a derivative thereof, a mutant thereof, a peptide thereof, a fragment thereof, an analog thereof or a mimetic thereof.

19. The method of claim 1, wherein the anticancer therapy is a small molecule inhibitor, molecularly targeted agent, antibody, chemical inhibitor, peptide, reactive oxygen species or free radical molecule, radiation therapy, chemotherapy or any other molecule capable of suppressing tumor cell growth.

20. The method of claim 19, wherein the anticancer therapy is an inhibitor of epithelial growth factor receptor (EGFR) selected from the group consisting of erlotinib, cetuximab, osimertinib, vandetanib, panitumumab, necitumumab, gefitinib and afatinib.

21-27. (canceled)

28. The method of claim 19, wherein the anticancer therapy is an inhibitor of mammalian target of rapamycin (mTOR) via direct inhibition or indirectly via binding of FK-binding protein 12, selected from the group consisting of omipalisib, dactolisib, pictilisib, idelalisib, buparlisib, torins, rapamycin, everolimus and temsirolimus.

29. The method of claim 19, wherein the anticancer therapy is an inhibitor of phosphoinositide 3-kinases (PI3K)/mTOR selected from the group consisting of omipalisib, dactolisib, pictilisib, idelalisib and buparlisib, 3 methyl adenine, wortmannin.

30. The method of claim 19, wherein the anticancer therapy is an inhibitor of protein kinase B (AKT) selected from the group consisting of uprosertib, MK-2206, ipatasertib, capivasertib and ARQ092.

31. (canceled)

32. The method of claim 19, wherein the anticancer therapy is an inhibitor of RAF proteins selected from the group consisting of regorafenib, sorafenib, dabrafenib and vemurafenib.

33. The method of claim 19, wherein the anticancer therapy is an inhibitor of mitogen-activated protein kinase kinase (MEK) selected from the group consisting of cobimetinib and trametinib.

34-53. (canceled)

54. The method of claim 1, wherein the biomarker is the localization, and/or level, and/or state of a molecule, and/or organelle.

55. The method of claim 54, wherein the molecule being measured is a protein or a nucleic acid.

56. The method of claim 54, wherein the state of the molecule being measured is phosphorylation, acylation, alkylation, amidation, glypiation, glycation, glycosylation, ubiquitination, degradation product(s), truncation, mutation status, or binding of the molecule(s) to promoters.

57. The method of claim 54, wherein the localization of the molecule being measured is extracellular or cellular, wherein cellular localization comprises intracellular, compartmentalized, nuclear or nucleoli, or membrane bound, wherein compartmentalized localization comprises Golgi, endoplasmic reticulum, lysosomal, endosomal, exosomal, mitochondrial, vacuole, and cytosolic localization, and wherein membrane bound includes plasma, nuclear, or other organelle membranes.

58. The method of claim 54, wherein the state of the organelle being observed is nuclear vacuolation/nuclear autophagy, or mitophagy.

59-77. (canceled)

78. The method of claim 1, wherein the biomarker(s) are measured using immunoassays, multiplexed assays, PCR, transcription factor assays, nucleic acid or sequencing/mutation testing, cell survival and cell viability assay.

79. The method of claim 78, wherein the immunoassay is selected from the group consisting of western blot, dot blot, ELISA, immunohistochemistry, immunocytochemistry, and immunofluorescence.

80. The method of claim 78, wherein the multiplexed assay is selected from the group consisting of flow cytometry, microarrays, and bead-based Luminex multiplex assays.

81. The method of claim 78, wherein the PCR is selected from the group consisting of qPCR, RT-PCR, real-time PCR and endpoint PCR.

82. The method of claim 78, wherein the transcription factor identification assay is selected from the group consisting of protein arrays, chromatin immunoprecipitation (CHIP) and CHIP-seq assays, DNA precipitation and DIP-seq assays, microsphere assays, DNAse sensitivity and gel shift assays.

83. The method of claim 78, wherein the nucleic acid or sequencing/mutation testing is selected from the group consisting of all forms of sequencing DNA and RNA molecules, whole genome, exome, or specific genes only, including massively parallel signature sequencing (MPSS), 454 pyrosequencing, Illumina (Solexa) sequencing, SOLiD sequencing, Ion Torrent semiconductor sequencing, Heliscope single molecule sequencing, single molecule real-time (SMRT) sequencing, sequencing by hybridization, and sequencing with mass spectrometry.

84. (canceled)

85. The method of claim 1, wherein the biomarker or panel of biomarkers is used to predict the likelihood of response, and wherein the biomarker or panel of biomarker are selected from the group consisting of EGFR, ALK, VEGF A, VEGF B, VEGFR, VEGFR1, VEGFR2, VEGFR3, KIT, HER2, CDK4, CDK6, PARP, mTOR, PI3K, AKT1, AKT2, AKT3, n-RAS, k-RAS, c-RAS, a-RAF, b-RAF, c-RAF, MEK1, MEK2, ERK1, ERK2, PDGFRα or PDGFRβ, MET, RET, ROS1, PIGF, PTCH, Smoothened, RANKL, and B4GALNT1, PD-1, PD-L1, PD-L2, STAT1, STAT3, STAT5, HER2, EGFR3, EGFR4, IGF1R, MET, KIT, RET, PDGFR, VEGFR, ALK, BCR-ABL, PTEN, PDK1, S6, p70 S6-Kinase, CREB, GSK3B, mTORC1 and mTORC2, Src, Fak, Ras, Raf, Mek, Erk, CREB, Sos-1, SHC, NFkB, cMyc, ELK-1, c-Fos and c-Jun, caspases 3, 6, 7, 8, 9, PARP, cytochrome c, Bim, Bad, Bax, Bcl-2, Bcl-xL, and Mcl-1, p27 Kip1, cyclin A, cyclin E, cyclin D, cyclin B, CDK1, CDK2, CDK4, CDK6, Cdc2, p16, p21, p14, p53, Ki67, PCNA, LC3I, LC3II, LC3/LC3I/LC3II ratios, MLKL, AMPK, p62/SQSTM1, ATG5-12 complex, ATG13, Vps34, AMBRA-1 and UVRAG, GABA receptor-associated protein like 1, syntaxin-17, LAMP1, LAMP2, LAMP2B, p38, Beclin1, ATM, UNC-51-like kinase-1, -2, and -3, Snail, Slug, Twist, ZEB, vimentin, vinculin, HMGB1, CD133, CD44, ABCG2, ALDH1A1, Hif1a, Hif2a, Brdu/EdU incorporation and CFSE staining, autophagosomes, autophagolysosomes, beta galactosidase, blebbing, Hoechest staining, annexin V staining, propidium iodide staining, WST8/MTT type assays, patient race, gender and age or age-related biomarkers comprising follicle-stimulating hormone, biopsy type, tumor stage, tumor type and/or histological categorization including cell cycle status, cell type and differentiation status of the sample.

86-90. (canceled)
